(19) United States
(12) Patent Application Publication
(10) Pub. No.: US 2026/0207165 A1
(43) Pub. Date: Jul. 23, 2026

(54) TONOMETER DEVICES AND METHODS

(71) Applicant: University of Pittsburgh - Of the Commonwealth System of Higher Education, Pittsburgh, PA (US)

(72) Inventors: Piervincenzo Rizzo, Pittsburgh, PA (US); Madison Hodgson, Saint Charles, IL (US); Samuel J. Dickerson, West View, PA (US)

(73) Assignee: University of Pittsburgh - Of the Commonwealth System of Higher Education, Pittsburgh, PA (US)

(21) Appl. No.: 19/455,601

(22) Filed: Jan. 21, 2026

Related U.S. Application Data
(60) Provisional application No. 63/747,839, filed on Jan. 21, 2025.

(51) Int. Cl. A61B 8/10; A61B 8/00

(52) U.S. Cl. CPC A61B 8/10; A61B 8/4254; A61B 8/4472; A61B 8/4494; A61B 8/5223; A61B 8/5269

(57) ABSTRACT

Methods include receiving tonometry data from a tonometer device, the data including time domain and frequency domain information, and estimating an intraocular pressure of an eye by processing the tonometry data through a machine learning model trained on pre-existing tonometry data and intraocular pressure data associated with the pre-existing tonometry data. Additional methods and related tonometers are disclosed.

### CROSS REFERENCE TO RELATED APPLICATION

This application claims the benefit of U.S. Provisional Patent Application No. 63/747,839, filed Jan. 21, 2025, and is incorporated by reference herein.

### ACKNOWLEDGMENT OF GOVERNMENT SUPPORT

This invention was made with government support under 2014389 awarded by the National Science Foundation. The government has certain rights in the invention.

### FIELD

The field is tonometry.

### BACKGROUND

Glaucoma is an age-related disease affecting the optic nerve and is the second leading cause of blindness in the world. Eye pressure is known to be a major risk factor for glaucoma. When the balance between the fluid production and drainage inside the eye is abnormal the intraocular pressure (IOP) increases, raising the risk of developing glaucoma.

In the U.S., nearly 9 million visits are made each year for the diagnosis or treatment of glaucoma but still, a significant fraction of glaucoma cases remains undiagnosed because the symptoms do not appear until significant damage occurs to the eye. According to the National Eye Institute (NEI): (1) women are more affected than men (61% vs. 39%); (2) the annual cost to the government is over $1.5B in health care expenditures, lost income tax revenues, and Social Security benefits; (3) by 2050 the number of people in the U.S. with glaucoma will almost triple. Worldwide, glaucoma affect ~4% of the population and 70+ million people have the disease without knowing it.

The measurement of IOP is the cornerstone of the diagnosis and management of glaucoma, as the elevated value of this pressure is the only risk factor that can be modified by proper therapy or surgical intervention. Unfortunately, IOP follows a circadian rhythm and fluctuates throughout the day. For this reason, a single office-based measurement is typically insufficient to discover daily changes and spikes, nor can they demonstrate the effect of medication or patients' compliance to a given therapy. Similar to diabetics measuring blood glucose levels, clinical evidence suggests that multiple daily measurements would be beneficial. However, this is possible only with an off-the-counter hand-held device that patients of any literacy and fair dexterity can self-administer. To satisfy these characteristics, the IOP measurement device should be easy-to-use, inexpensive, and not require sterilization or topical anesthesia, by way of example. Devices can further benefit from various features and capabilities that can improve measurement accuracy. Thus, a need remain for improved devices, such as ones that can include one or more of these advantages, and which are not currently available to glaucoma patients.

### SUMMARY

According to aspects of the disclosed technology, apparatus and methods measure intraocular pressure of an eye through stress waves, e.g., transmitted through an eyelid. Some examples can provide an indication of suitable tonometer positioning/application of the tonometer device relative to the eye, to improve measurement accuracy and repeatability. Some examples can allow for personalized predictions for a patient by using corneal characteristics such as thickness as an input to a prediction model. Prediction capability can include processing measurements, such as spectrograms, through a machine learning model, such as a convolutional neural network.

According to an aspect of the disclosed technology, methods include receiving tonometry data from a tonometer device, the data including time domain and frequency domain information, and estimating an intraocular pressure of an eye by processing the tonometry data through a machine learning model trained on pre-existing tonometry data and intraocular pressure data associated with the pre-existing tonometry data. In some examples, the tonometry data comprises stress wave data and the pre-existing tonometry data comprises pre-existing stress wave data. In some examples, the pre-existing tonometry data includes pre-existing corneal thickness data, wherein tonometry data includes corneal thickness measurement data associated with the eye, wherein the estimating includes processing the tonometry data including the corneal thickness measurement data associated with the eye through the trained machine learning model. In some examples, the pre-existing tonometry data includes pre-existing eyelid thickness data, wherein tonometry data includes eyelid thickness measurement data associated with the eye, wherein the estimating includes processing the tonometry data including the eyelid thickness measurement data associated with the eye through the trained machine learning model. In some examples, the trained machine learning model comprises a convolutional neural network. In some examples, the time and frequency domain information comprise one or more spectrograms. Some examples include positioning an end of a tonometer device proximate the eye, directing one or more incident tonometer waves to the eye along a wave carrier and receiving one or more return tonometer waves from the eye, detecting at least the one or more return tonometer waves with a tonometer sensor to produce a tonometer signal associated with the tonometry data. In some examples, the directing comprises directing one or more incident solitary stress waves to the eye, and the detecting comprises detecting at least a primary reflected solitary stress wave and a secondary solitary stress wave for each incident solitary stress wave directed to the eye. In some examples, the directing comprises producing the one or more incident solitary waves in the wave carrier with an actuator. In some examples, the actuator comprises a striker. In some examples, the positioning comprises contacting the end of the tonometer device to an eyelid of the eye. In some examples, the end comprises a flexible membrane configured to directly contact the eye or eyelid of the eye. In some examples, the end comprises a retained end particle of a chain of particles comprising the wave carrier, wherein the end particle is configured to directly contact the eye or eyelid of the eye. In some examples, the directing comprises directing the one or more incident tonometer waves to the eye through the eyelid. Some examples include reducing electrical reflections that deteriorate the tonometer signal received by a microcontroller of the tonometer device by providing an impedance matching between the microcontroller and an electrical circuit coupling the tonometer sensor to the microcontroller. Some examples include determining a suitability of an alignment of the tonometer device in relation to the eye before performing a tonometer measurement, by detecting an orientation of the tonometer device in relation to Earth's gravitational field using an inclinometer of the tonometer device. In some examples, the determining the suitability of the alignment comprises determining whether wave carrying and striking components of the tonometer device are aligned within a range parallel to Earth's gravitational field. Some examples include preventing the performing of a measurement where the alignment is determined to be not suitable. Some examples include providing an audio and/or visual indication before and/or after the alignment is determined to be suitable. Some examples include training the machine learning model on the pre-existing tonometry data and the intraocular pressure data associated with the pre-existing tonometry data.

According to another aspect of the disclosed technology apparatus include tonometers configured to perform the methods described herein.

According to another aspect of the disclosed technology, apparatus include a wave carrier configured to propagate one or more incident stress waves to an eye, a housing configured to support the wave carrier, a sensor coupled to the wave carrier and configured to detect one or more return stress waves propagating along the wave carrier from the eye, and one or more processors configured to receive stress wave tonometry data from the sensor, the data including time domain and frequency domain information, wherein the at least one of the one or more processors is configured estimate an intraocular pressure of the eye based on both the time domain and frequency domain information. In some examples, at least one of the one or more processors is configured to estimate the intraocular pressure by processing the stress wave tonometry data through a machine learning model trained on pre-existing tonometry data and intraocular pressure data associated with the pre-existing tonometry data. In some examples, the wave carrier comprises a particle array, wherein the particle array comprises a plurality of adjacently arranged loosely coupled particles that propagate the incident and return stress waves from one particle to the next. Some examples include a particle array compressive member coupled to at least one of the particles to provide a compression for the particle array that contact among the particles. In some examples, the particles have spherical, cylindrical, or elliptical shape, or a mix of shapes, and are made of PTFE, steel, or another material having an elastic modulus between 0.01 and 200 GPa. In some examples, the sensor comprises a magnetic coil encircling at least a portion of at least one of the particles or a piezoelectric transducer embedded in at least one of the particles. In some examples, the sensor comprises a stress wave sensor. Some examples include a retaining support configured to retain the wave carrier in the housing and to allow an end of the wave carrier to become removably coupled to the eyelid of the eye. In some examples, the retaining support comprises a membrane attached to the housing. In some examples, the retaining support comprises an arcuate or circular ridge. In some examples, the retaining support is configured to allow an end of the wave carrier to directly contact the eyelid. Some examples include an actuator coupled to the particle array and configured to produce the incident stress wave in the wave carrier. Some examples include driving circuitry configured to drive the actuator wherein the driving circuitry includes delay circuitry configured to reduce a sampling error, and filter circuitry configured to filter stress wave data detected by the sensor. Some examples include circuitry configured to wirelessly transmit the filtered stress wave data to a separate computing device. In some examples, the actuator comprises a solenoid configured to raise a striker particle and to drop the striker particle from a height. Some examples include a digitizer coupled to the sensor and configured to digitize the detected return stress wave to form a digitized return solitary wave signal, a processor coupled to the digitizer and function generator, and a memory coupled to the processor and configured with instructions executable by the processor for controlling the generation of the incident stress wave in the wave carrier. In some examples, the memory is further configured with instructions for determining an intraocular pressure of an eye based on one or more characteristics of the digitized return stress wave signal. Some examples include a wireless communication node coupled to the processor and configured to communicate data describing the digitized return stress wave signal to an external signal processing device.

According to another aspect of the disclosed technology, methods include directing an incident stress wave along a wave carrier coupled to an eye, detecting at least one return stress wave propagating along the wave carrier from the eye, and producing a detected stress wave signal including time domain and frequency domain information. Some examples include estimating an intraocular pressure of the eye by processing the stress wave tonometry data of the stress wave signal through a machine learning model trained on pre-existing tonometry data. Some examples include estimating an intraocular pressure by comparing characteristics of the stress wave signal to a relationship between a time of return stress wave time of flight and/or a ratio of incident and detected wave amplitudes and a correlated intraocular pressure.

According to another aspect of the disclosed technology, computer-readable media include stored instructions which, when executed by one or more computing devices, cause the computing devices to estimate intraocular pressure according any of the apparatus and methods described herein. Some examples include stored instructions causing the computing devices to direct an actuator to produce an incident stress wave along a wave carrier coupled to the eye, and to store the stress wave data including data from a detection signal received in response to the actuating.

According to another aspect of the disclosed technology, tonometers can include a tonometer wave carrier arranged to propagate one or more waves to an eye, a tonometer sensor coupled to the wave carrier to detect characteristics of one or more return waves received in response to the one or more waves that propagate to the eye, and a microcontroller circuit electrically coupled to the sensor to receive an electrical signal from the sensor, wherein the electrical signal has characteristics based on the detected one or more return waves, wherein the electrical coupling between the microcontroller circuit and the sensor is impedance matched to reduce electrical reflections that reduce a quality of the electrical signal received by the microcontroller. In some examples, the microcontroller circuit includes a low pass filter and an analog to digital converter and the sensor includes a sensing element and wiring coupling the sensor to the microcontroller circuit.

According to another aspect of the disclosed technology, apparatus include a wave carrier configured to propagate one or more incident waves to an eye, a housing configured to support the wave carrier and be held by a user to measure an intraocular pressure of the eye, a sensor coupled to the wave carrier and configured to detect one or more return waves propagating along the wave carrier from the eye, and a sensor coupled to the wave carrier and housing and configured to detect an orientation of the wave carrier in relation to Earth's gravitational field, wherein the detected orientation is used to provide the user with an indication of suitability and/or non-suitability of the orientation for intraocular pressure measurement.

The foregoing and other objects, features, and advantages of the disclosed technology will become more apparent from the following detailed description, which proceeds with reference to the accompanying figures.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic of a solitary wave tonometry operation.

FIG. 2 is a graph of dynamic force with respect to time for a tonometry measurement.

FIG. 3 is a schematic of an example tonometer positioned proximate an eye to be tested.

FIG. 4 is a schematic of an example tonometry controller and sensor system that is impedance matched to improve signal detection.

FIG. 5 is a flowchart of example methods of performing tonometry measurements.

FIG. 6 is a flowchart of an example artificial neural network training and trained estimation.

FIG. 7 is a schematic of an example tonometer being positioned for measurement.

FIG. 8 is a schematic of an example tonometry system.

FIGS. 9A-9B show spectrograms and a graph of prediction results of a trained neural network.

FIG. 10 is a schematic of an example computing device.

FIG. 11 is an image of a PCB next to a schematic of a tonometry arrangement coupled to the PCB.

FIG. 12 is a graph of stress wave amplitude with respect to time.

FIG. 13 is a graph of stress vs. strain measurements from an experiment.

FIGS. 14A-14C are a cross-section of a model cornea, its 3D rendering, and an image of a corresponding test specimen.

FIGS. 15A-15C are color images of STFT stress wave spectrograms.

FIG. 16 is a schematic of a convolutional neural network.

FIG. 17 is a graph of average stress wave time of flight (ToF) with respect to pressure for test specimens with different corneal thicknesses.

FIG. 18 is a graph of averaged time of flight with respect to corneal thickness for three different loading ramp pressures.

FIG. 19 is a plot of time of flight with respect to pressure for three different corneal thicknesses.

FIG. 20 is a confusion matrix associated with convolutional neural network predictive performance for corneal thickness.

FIG. 21 is a schematic of an example tonometry system.

FIG. 22 is a schematic of an example tonometry system arrangement.

FIG. 23 is a side cross-sectional schematic of an example tonometry device.

FIG. 24 is a perspective schematic of an example tonometry system.

FIG. 25 is a schematic of an example tonometer configured to operate wirelessly with a mobile device.

FIG. 26 is an image of an example PCB that can be used in example tonometers.

FIG. 27 is a schematic of an example circuit that can be used in tonometers.

### DETAILED DESCRIPTION

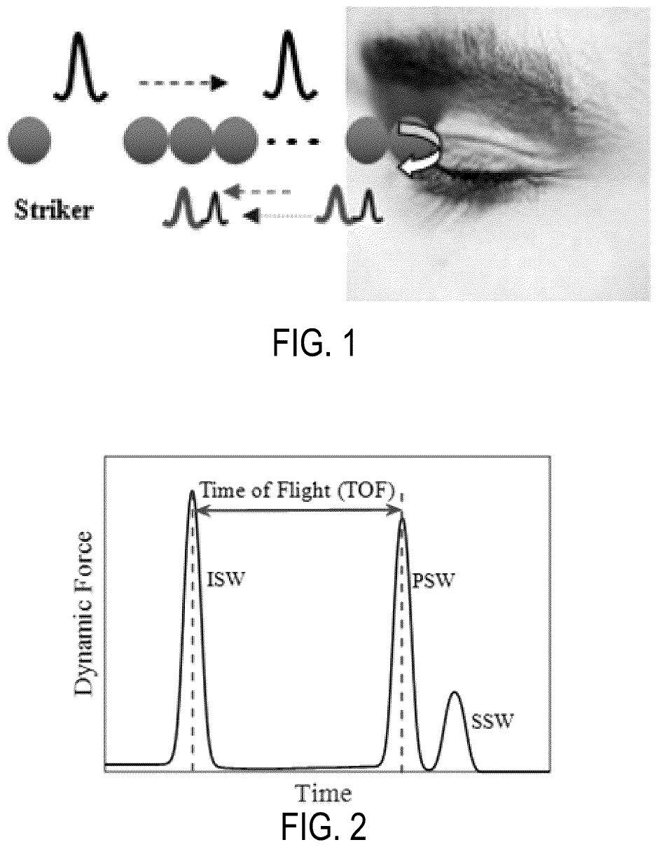

Examples herein can enable the early detection and the proper treatment of glaucoma by enabling frequent measurements of the intraocular pressure (IOP). An engineering principle associated with various representative examples is shown in FIG. 1. A medium that can propagate mechanical waves (stress waves, solitary waves, etc,), such as a chain of a few mm small particles, can be in communication with the lid of the eye for which IOP is to be estimated. An incident mechanical wave, such as a solitary wave (ISW), is induced at one end (such as mechanically and/or electrically, e.g., with a striker or actuator), propagates along the chain, and reaches the eye (e.g., by propagating through an eyelid); here the single pulse is reflected back to the chain originating one or more reflected waves. Example tonometers using incident solitary waves and detected return waves are shown in U.S. Pat. No. 11,957,413 to Rizzo et al., which is incorporated by reference herein. FIG. 2 are example amplitude traces of the ISW (moving towards the eyelid) and the first of typically two reflected pulses, with the two reflected pulses hereinafter being referred to as the primary and secondary reflected waves (PSW and SSW), generated at the interface with the eyelid. The amplitude and travel time of the reflected pulses can be dependent on the eye pressure. In some examples, in addition to or as an alternative to solitary waves, other characteristics of waves directed to and/or received from the eye can be used to estimate intraocular pressure. In some examples, the dependence can occur irrespective of the cornea thickness and/or eyelid stiffness (or an IOP dependence on cornea thickness and/or eyelid stiffness can be controlled through calibration). In further examples, cornea thickness can be measured and used in the estimation process. Representative device embodiments can be placed in contact with the eyelid of the eye to be measured, thereby enabling any patient to self-administer a tonometry test to capture, store, and transmit wirelessly the physiological state of their eye pressure. In further examples, a device surface can directly contact the sclera.

### Representative Examples

Elevated IOP is one of the major risk factors for the development and progression of glaucoma. [Heijl, A., Leske, M. C., Bengtsson, B., Hyman, L., Bengtsson, B., & Hussein, M. (2002). Reduction of intraocular pressure and glaucoma progression: results from the Early Manifest Glaucoma Trial. Archives of ophthalmology, 120 (10), 1268-1279]. Accurate assessment of IOP is important because elevated IOP is the only risk factor that can be modified by therapeutic interventions [Lee, T. E., Yoo, C., Lin, S. C., & Kim, Y. Y. (2015). Effect of different head positions in lateral decubitus posture on intraocular pressure in treated patients with open-angle glaucoma. American journal of ophthalmology, 160 (5), 929-936]. The fact that IOP follows a circadian rhythm and is also subjected to spontaneous changes throughout the day, makes office-based single measurements neither sufficient to discover daily changes and spikes, nor valid to demonstrate the effect of medication or patients' compliance to a given therapy. As such, frequent daily measurements would be ideal, similar to diabetics measuring blood glucose levels. However, this is possible only with an off-the-counter hand-held not-sticking device that patients of any literacy and fair dexterity can self-administer. To satisfy these characteristics, the device should be easy-to-use, inexpensive, and should not require sterilization or topical anesthesia. Devices could also further benefit the user by providing an indication that suitable conditions are present for an accurate measurement, providing robust measurement signal data even where noise may be present, providing measurements based on solitary wave and/or other wave phenomena, and/or provide sophisticated and highly accurate measurements (e.g., with machine learning models) which, in some examples, can be specifically tailored to characteristics of the user's eye (such as corneal thickness). Exemplary devices and methods can contribute to development of a new generation of instruments to be used in eye care.

### Tonometry

Methods of measuring IOP can be clustered in three large groups: palpation, manometry, and tonometry [1]. Palpation is the oldest, simplest, least expensive, and least accurate method. It consists of displacing the redundant skin of the upper eyelid and balloting alternatively the central meridian of the globe with the tips of each index finger [1]. Manometry is the most precise and the most invasive approach because a hollow needle is surgically inserted into the anterior chamber. Manometry provides the reference pressure by which all other methods should be judged. It is mainly used in laboratory and its use in living human eyes is restricted to eyes undergoing enucleation or intraocular surgery [1]. Tonometry is based on the relationship between IOP and the force necessary to deform the cornea by a given amount [2]. Among the three groups, tonometry is the preferred approach because it is not invasive as manometry and is more accurate than palpation.

Tonometers can be sub-grouped in applanation, rebound, and indentation, and correspond to the physical principles of tonometers applied in clinical practice today. The gold standard for measuring IOP is the Goldmann Applanation Tonometer (GAT) against which any other methods are judged and compared. GAT is based on the Imbert-Fick principle IOP=F/A, which states that the IOP is proportional to the force F needed to applanate a pre-defined area A [3,4]. However, this law is only applicable to an infinitely thin membrane perfectly elastic, dry, and flexible [3-5]. In reality, none of these assumptions applies to applanation of the cornea, which has variable curvature, has finite thickness, is not perfectly elastic, is coated by the tear film, and is a small part of the overall larger-diameter eyeball, which is connected via the limbus to the sclera. GAT requires the use of a drop of anesthetic and fluorescein, must be proctored by a health care professional, and must be administered with the patient in a sitting position [5].

Rebound tonometers are ballistic devices that measure the return-bounce motion of an object impacting the cornea [1]. ICare is the most widely used rebound tonometer. It mounts a single-use probe that exchanged after every patient; the probe is propelled against the cornea, impacts with it and rebounds from the eye. Individual measurements are digitally displayed, and after six consecutive measurements the average and the standard deviation are given [6]. On thick corneas, Icare overestimates IOP even more than GAT. Intersessional repeatability of IOP taken with the Icare is poorer than with GAT. Icare also developed Icare HOME for self-tonometry. However, a 2016 study [7] concluded that: “Not all participants could learn how to use the Icare HOME device, but for those who could, [ . . . ] nearly 1 in 6 individuals may fail to certify in use of the device based on large differences in IOP when comparing GAT with the Icare HOME measurements”. Finally, this device was not approved by the FDA.

TonoPen is a hybrid applanation/indentation system in which a tip is covered by a disposable latex cover and applied perpendicularly to indent an anesthetized cornea. Owing to the requirements for a localized anesthesia, this device cannot be proctored home and need to be administered by an eye care professional. Each measurement requires several applanations. An acceptable applanation is indicated by an audible click after contact with the cornea. A microprocessor averages the acceptable waveforms and gives a digital readout of IOP. TonoPen gives higher readings than GAT, and above 21 mmHg it underestimates GAT readings.

The tonometer TGDc-01 is a device designed to measure the IOP through the eyelids without anesthesia. The movement of a small rod falling freely onto the eyelid surface is measured. Individual measurements are displayed digitally. Three measurements are usually performed [6]. Troost et al. proved that TGDc-01 underestimates the IOP when compared with GAT [1,8-10]. Deviations between the TGDc-01 and the GAT were found to be clinically relevant and therefore TGDc-01 could not be considered as an alternative to GAT [7-6]. There is also the uncomfortable sensation for the patient of the rod tapping the eyelid.

Yung et al. [11] reviewed the technologies for self-tonometry and for continuous monitoring of IOP currently undergoing development and clinical trials: portable devices, contact lenses, and telemetry using implantable pressure sensors. Besides the invasive nature of these solutions, some of their conclusions were: “[ . . . ], no effective method of 24-hour IOP monitoring currently exists outside of office visits. Current portable devices for IOP measurement have not been shown to be reliable for home use by patients, and have not yet yielded accurate results compared to GAT. These devices are still at the research stage and do not have any commercial name yet.

Various tonometry examples of the disclosed technology herein may resemble the rebound tonometry in some respects. However, representative examples herein do not require tapping, impacting, or applanating the cornea, do not require topical anesthesia, and/or do not require trained health care professionals to make reliable measurements.

### Tonometry References [1]-[11] Referenced Above

1 C. Kniestedt, O. Punjabi, S. Lin, and R. L. Stamper (2008). “Tonometry Through the Ages”, Survey of Ophthalmology, 53 (6), 568-591.
2 European Glaucoma Society, Terminology and guidelines for glaucoma, 4th Edition, June 2014.
3 Goldmann H (1957): Applanation tonometry. New York. Josiah Macy, Jr. Foundation
4 Goldmann H, Schmidt T (1957) “Applanation Tonometry,” Ophthalmologica, 134 (4), 221-242.
5 Jóhannesson, G. (2011). Intraocular pressure—clinical aspects and new measurement methods, Ph.D. dissertation Umea University, Sweden.
6 Liane H. Van Der Jagt, Nomdo M. Jansonius (2005). “Three portable tonometers, the TGDc-01, the ICARE and the Tonopen XL, compared with each other and with Goldmann applanation tonometry,” Ophthalmic and Physiological Optics, 25 (5), 429-435.
7 Mudie, L. I., LaBarre, S., Varadaraj, V., Karakus, S., Onnela, J., Munoz, B., and Friedman, D. S. (2016). The Icare HOME (TA022) Study: Performance of an Intraocular Pressure Measuring Device for Self-Tonometry by Glaucoma Patients. Ophthalmology.
8 Dabasia, P. L., Lawrenson, J. G., and Murdoch, I. E. (2015). Evaluation of a new rebound tonometer for self-measurement of intraocular pressure. British Journal of Ophthalmology,
9 Müller A, Godenschweger L, Lang G E, et al. (2004). “Prospective comparison of the new indentation tonometer TGdC-01, the non-contact tonometer PT100 and the conventional Goldmann applanation tonometer,” Klin Monatsbl Augenheilkd, 221, 762-768.
10 Troost A, Specht K, Krummenauer F, et al. (2005). “Deviations between transpalpebral tonometry using TGDc-01 and Goldmann applanation tonometry depending on the IOP level,” Graefes Arch Clin Exp Ophthalmol, 243, 853-858.
11 Yung, E., Trubnik, V., and Katz, L. J. (2014). An overview of home tonometry and telemetry for intraocular pressure monitoring in humans. Graefe's Archive for Clinical and Experimental Ophthalmology, 252 (8), 1179-1188.

### Solitary Wave-Based Tonometry Measurement Models and Experiments

The following description relates to the article by Nasrollahi and Rizzo “Modeling a New Dynamic Approach to Measure Intraocular Pressure with Solitary Waves,” Journal of the Mechanical Behavior of Biomedical Materials, 103, March 2020, 103534, https://doi.org/10.1016/j.jmbbm.2019.103534, and which is incorporated by reference herein.

Some examples of disclosed tonometers can incorporate engineering principles schematized in FIG. 1. A short granular chain made of a few mm spherical particles, hereinafter referred to as the chain, is in point-contact with the lid of the eye to be diagnosed. The particles support the propagation of highly nonlinear solitary waves (HNSWs), which are a special kind of stress waves fundamentally different than those waves typically encountered in acoustics and ultrasound. Those waves are characterized by having a return force linearly dependent on the displacement. HNSWs are instead nonlinear: the return force F is nonlinearly proportional to the displacement from equilibrium according to the Hertz's law F=A_{b}δ^{3/2}. Here δ is the indentation between two adjacent identical interacting beads, and A_{b} is the contact stiffness equal to [E_{b}(2R_{b})^{0.5}]/[3(1−ν_{b}²)] where E_{b}, R_{b}, and ν_{b} are the beads modulus, radius, and Poisson's ratio, respectively. HNSWs are also unique with respect to conventional linear waves because their intrinsic tunability makes them useful for a wide range of engineering applications, including but not limited to nondestructive evaluation (NDE), energy harvesting, and impact mitigation. A typical time waveform of these pulses is shown in FIG. 2 where an incident solitary wave (ISW) is induced at one end by the mechanical impact of a striker. The incident wave propagates along the chain of spherical particles and reaches the eyelid. This single pulse can give rise to two reflected pulses, the primary and the secondary reflected solitary waves (PSW and SSW). The research hypothesis investigated in a feasibility study was that the amplitude and time-of-flight (ToF) of these reflected pulses are monotonically dependent on the eye pressure. However, in various tonometry device examples herein, wave features that can be included in the analysis to identify or estimate IOP can include but are not limited to amplitudes of the three waves (ISW, PSW, SSW), the time of flight of the PSW and/or SSW, the width at half amplitude of each of the three waves, and any declination in terms of their ratios or product, such as the ratio of the amplitude of the PSW to the amplitude of the ISW or the product of the two amplitudes, by way of example. Further, many examples can incorporate other characteristics of solitary or other waves propagating to and/or from the eye, such as spectral characteristics.

Recently, HNSWs were used to characterize tennis balls and their internal pressure. A finite element model was modified and coupled to a discrete particle model to describe the dynamic interplay between the solitary waves and sub-millimeter soft material (the human cornea) under varying pressure. Parameters such as the internal pressure and the geometric and mechanical properties of the chain were varied in order to investigate the effect of these characteristics on the sensitivity of new tonometer instruments.

In analyzing underlying engineering principles and applications to ophthalmology, the mechanical interaction between solitary waves and thin walled soft materials was investigated. The ability of the waves to be used to measure internal pressure was assessed and the feasibility of solitary wave-based tonometer devices was also explored. Further examples were developed that can provide non-invasive tonometry applications based on solitary waves.

The following description presents a finite element formulation developed to predict the dynamical interaction between the waves and the cornea. The model was adapted from existing models to measure the internal pressure of tennis balls in order to account for the geometric and mechanical properties of the cornea. A spring-mass model is coupled to the finite element formulation to describe the propagation of the solitary waves along the chain. Also, a numerical setup was described to quantify the effects of the internal pressure on some selected features of the solitary waves, along with related numerical results.

A four-node quadrilateral axisymmetric element was used. Each node had one degree of freedom u in the radial direction r(ζ,n) (u₁, u₂, u₃, u₄) and one degree of freedom w in the vertical direction z(ζ,n) (w₁, w₂, w₃, w₄). Due to the axisymmetric nature of the problem, the Cauchy stress vector and the strain vector were σ=[or σᵣ σ_{z} σ_{θ} τ_{rz}]^{T} and ε=[εᵣ ε_{z} ε_{θ} σ_{rz}]^{T}, respectively. This implied that for each element, there were three normal stresses/strains in the radial, vertical, and angular directions and one shear stress/strain in the radial-vertical direction). The material stiffness matrix K^{mat} of the element was determined:

$${K}^{mat} = 2 π {\int }_{- 1}^{1} {\int }_{- 1}^{1} ( {B}^{T} ( ζ , η ) \cdot C \cdot B ( ζ , η ) r ( ζ , η ) det J ( {ζ}_{i} , {η}_{j} ) ) d ζ d η ≅ 2 π {\sum }_{i = 1}^{m} {\sum }_{j = 1}^{n} {w}_{ij} {B}^{T} ( {ζ}_{i} , {η}_{j} ) \cdot C \cdot B ( {ζ}_{i} , {η}_{j} ) r ( {ζ}_{i} , {η}_{j} ) det J ( {ζ}_{i} , {η}_{j} ) \qquad ( 1 )$$

where m and n is the number of Gaussian points in ζ and η directions, respectively, used in the numerical integration, wᵢⱼ are the weight coefficients, J(ζ, n) is the Jacobian matrix, and B(ζ, n) is the strain-displacement matrix used to compute the strains ε at any point inside the element using the nodal displacement vector d as:

$$ε = {B}^{T} \cdot d \qquad ( 2 )$$

Furthermore, Eq. (1) contains the stress-strain matrix C, which for a linear-elastic isotropic material equals to:

$$C = \frac{E}{( 1 + v ) ( 1 - 2 v )} [ \begin{matrix}1 - v & v & v & 0 \\ v & 1 - v & v & 0 \\ v & v & 1 - v & 0 \\ 0 & 0 & 0 & ( 1 - 2 v ) / 2\end{matrix} ] \qquad ( 3 )$$

where E is the Young's modulus and vis the Poisson's ratio of the cornea. In some examples, the modulus of the human cornea can be considered as a linear function of the IOP As such, Eq. (3) takes into account the internal pressure of the eye by updating the value of the Young's modulus of the cornea. However, this does not generally represent an impediment in a clinical setting where the IOP is the parameter to be measured. In various examples, other relations between IOP and solitary wave characteristics can be obtained and used to make IOP measurements with solitary waves.

The stress σ and the consequent strain ε generated by the internal pressure were treated as initial parameters in the eye. Thus, the geometric K^{geo} and the total stiffness K were proportional to the internal pressure. The geometric nonlinear stiffness matrix K^{geo} was given by [7]:

$${K}^{geo} = 2 π {\int }_{- 1}^{1} {\int }_{- 1}^{1} ( {β}^{T} ( ζ , η ) \cdot σ \cdot β ( ζ , η ) r ( ζ , η ) det J ( {ζ}_{i} , {η}_{j} ) ) d ζ d η ≅ 2 π {\sum }_{i = 1}^{m} {\sum }_{j = 1}^{n} {w}_{ij} {β}^{T} ( {ζ}_{i} , {η}_{j} ) \cdot σ \cdot β ( {ζ}_{i} , {η}_{j} ) r ( {ζ}_{i} , {η}_{j} ) det J ( {ζ}_{i} , {η}_{j} ) \qquad ( 4 )$$

where β contains the derivatives of the shape functions. The total stiffness of the cornea was the sum of the material stiffness matrix and the geometric nonlinear stiffness matrix, i.e.:

$$K = {K}^{mat} + {K}^{geo} \qquad ( 5 )$$

Finally, the mass matrix M and the load vector f for each element were given by:

$$M = 2 πρ {\int }_{- 1}^{1} {\int }_{- 1}^{1} ( {N}^{T} ( ζ , η ) r ( ζ , η ) det J ( ζ , η ) ) d ζ d η ≅ 2 π p {\sum }_{i = 1}^{m} {\sum }_{j = 1}^{n} {w}_{ij} {N}^{T} ( {ζ}_{i} , {η}_{j} ) r ( {ζ}_{i} , {η}_{j} ) det J ( {ζ}_{i} , {η}_{j} ) \qquad ( 6 )$$

$$f = 2 π {\int }_{- 1}^{1} {\int }_{- 1}^{1} ( {N}^{T} ( ζ , η ) \{ \begin{matrix}{T}_{x} ( ζ , η ) \\ {T}_{y} ( ζ , η )\end{matrix} \} r ( ζ , η ) det J ( ζ , η ) ) d ζ d η ≅ 2 π {\sum }_{i = 1}^{m} {\sum }_{j = 1}^{n} {w}_{ij} {N}^{T} ( {ζ}_{i} , {η}_{j} ) \{ \begin{matrix}{T}_{x} ( {ζ}_{i} , {η}_{j} ) \\ {T}_{y} ( {ζ}_{i} , {η}_{j} )\end{matrix} \} r ( {ζ}_{i} , {η}_{j} ) det J ( {ζ}_{i} , {η}_{j} ) ) \qquad ( 7 )$$

where N(ζ, η) is the shape functions vector in isoparametric (natural) coordinates, ρ is the density of the material, Tₓ and T_{y} are the tractions along x and y directions, respectively, which can represent the components of the internal pressure along x and y, respectively, in some examples.

To obtain the stiffness and mass matrices as well as the load vector of the whole cornea, K, M and f were computed for each element of the mesh and then assembled using the connectivity matrix, formulated by implementing the advancing front method.

As stated above, the above finite element formulation was coupled to a discrete mass/spring model to predict the effect of the IOP on the propagation of the solitary waves inside the chain made of N spheres. The second Newton's law was applied to the displacement uᵢ(t) of the i^{th} particle of mass mb yielding to the following set of differential equations of motion:

$${\ddot{u}}_{1} ( t ) = {\frac{{A}_{b}}{{m}_{b}} [ {u}_{2} ( t ) - {u}_{1} ( t ) ]}_{+}^{3 / 2} - g \qquad ( 8 a )$$

$${\ddot{u}}_{1} ( t ) = {\frac{{A}_{b}}{{m}_{b}} [ {u}_{i + 1} ( t ) - {u}_{i} ( t ) ]}_{+}^{3 / 2} - {\frac{{A}_{b}}{{m}_{b}} [ {u}_{i} ( t ) - {u}_{i - 1} ( t ) ]}_{+}^{3 / 2} - g , i = 2 , 3 , … , N - 1 \qquad ( 8 b )$$

$${\ddot{u}}_{1} ( t ) = {\frac{{A}_{c}}{{m}_{b}} [ {u}_{M , c} ( t ) - {u}_{N} ( t ) ]}_{+}^{3 / 2} - {\frac{{A}_{b}}{{m}_{b}} [ {u}_{N} ( t ) - {u}_{N - 1} ( t ) ]}_{+}^{3 / 2} - g \qquad ( 8 c )$$

In Eq. (8), the first particle (i=1) represents the striker whose motion triggers the formation of the incident wave. The last particle (i=N) is instead the bead in contact with the eye to be evaluated. Furthermore, g is the gravity, [x]₊ means max (x,0), u_{Mc} is the displacement of the cornea along the direction of the wave propagation, and A_{c} is the contact stiffness at the cornea/bead interface. This Hertzian contact stiffness was obtained by dividing the magnitude of the load, applied at the contact point, to the corresponding displacement. Eq. (8) contains the Hertzian contact stiffness A_{b} between two adjacent beads that, as mentioned hereinabove, is equal to:

$${A}_{b} = \frac{{E}_{b} \sqrt{2 {R}_{b}}}{3 ( 1 - {v}_{b}^{2} )} \qquad ( 9 )$$

For the cornea, the equation of motion was computed as:

$$\ddot{u} ( t ) = {M}_{rg}^{- 1} \cdot {f}_{rg} ( t ) - ( {M}_{rg}^{- 1} \cdot {K}_{rg} ) \cdot u ( t ) \qquad ( 10 )$$

where M_{rg}, K_{rg}, and f_{rg}(t) are, respectively, the reduced global mass and stiffness matrices and the reduced global force vector, all obtained after applying the boundary conditions. f_{rg}(t) includes static force due to the internal pressure and dynamic force of the HNSW. Displacements of the beads and the cornea were obtained by solving simultaneously Eqs. (8) and (10). These displacements were replaced into the Hertz's contact law:

$${f}_{1} ( t ) = {{A}_{b} [ {u}_{2} ( t ) - {u}_{1} ( t ) ]}_{+}^{3 / 2} \qquad ( 11 a )$$

$$( 11 b )$$

$${f}_{i} = \frac{1}{2} ( {{A}_{b} [ {u}_{i + 1} ( t ) - {u}_{i} ( t ) ]}_{+}^{3 / 2} - {{A}_{b} [ {u}_{i} ( t ) - {u}_{i - 1} ( t ) ]}_{+}^{3 / 2} ) , i = 2 , 3 , … , N - 1$$

$${f}_{n} ( t ) = \frac{1}{2} ( {{A}_{c} [ {u}_{M , c} ( t ) - {u}_{N} ( t ) ]}_{+}^{3 / 2} - {{A}_{b} [ {u}_{N} ( t ) - {u}_{N - 1} ( t ) ]}_{+}^{3 / 2} ) \qquad ( 11 c )$$

to determine the dynamic force at each bead of the chain.

The cornea of healthy young adults (22-29 year-old) was considered. A circle sector of 7.8 mm radius and central angle equal to 120° was modeled. The geometry of the finite element model was adapted to the axisymmetric nature of the physical phenomena being investigated. The thickness, density and Poisson's ratio of the cornea were equal to 0.536 mm, 1000 kg/m3 and 0.49, respectively. The Young's modulus of the cornea can be understood as a function of the eye pressure. Ten IOPs were considered ranging from 12.75 mmHg (1700 Pa) to 30.00 mmHg (4000 Pa) at step of 1.725 mmHg (230 Pa). Across this range, the cornea's modulus varied between 90 kPa and 900 kPa. However, various modulus relations can depend on conditions and eye characteristics, and thus disclosed examples are not limited to the specific relations shown.

Mesh and the boundary conditions were selected and considered. An advancing-front method was coded in MATLAB to mesh the cornea. The mesh consisted of 320 elements, 80 elements along the arc length and 4 elements along the radial direction, i.e. across the thickness. A Gaussian elimination method was used for the static analysis of the cornea under internal pressure and a built-in simultaneous 4-5th-order Runge-Kutta command in MATLAB (ode45) was employed to analyze the propagation of the solitary pulses along the chain placed in contact with the cornea.

Four chains made of twenty particles were considered in order to find the characteristics (diameter and modulus) of the particles that would provide the highest sensitivity of the solitary waves to the IOP variation. Two particles diameter, namely d=1 mm and 2 mm, and two materials, namely stainless steel and polytetrafluoroethylene (PTFE), were considered. For the steel: E_{b}=200 GPa, ν_{b}=0.3, and ρ_{b}=7,850 kg/m³; for the PTFE: E_{b}=0.5 GPa, ν_{b}=0.46, and ρ_{b}=2,200 kg/m³. Using Eq. (11b) the force amplitude of the pulses traveling through the tenth particle was measured. In this feasibility study, the tonometer was assumed to be in the vertical position. To mimic the free fall of the striker 1 mm above the chain, the initial velocity of the topmost sphere was set equal to 0.14 m/s. The numerical sampling frequency was equal to 2 MHz.

Deformation of the cornea varied under four different internal pressures. The deformation under 12.75 mm Hg (1700 Pa) was the largest. This counterintuitive outcome is due to the increase of the Young's modulus with the internal pressure: as the cornea becomes stiffer with the increase in pressure, the deformation becomes smaller.

The chain was then placed on the strained cornea. The weight of the chain deformed the cornea further, but such deformation was about 4.5 μm for the 2 mm-PTFE beads case, i.e. much smaller than the one caused by the eye pressure. As such, the self-weight of the proposed tonometer has no adverse effects on the patients' eye.

As discussed above, in experiments, an incident wave was triggered by setting the initial velocity of the striker to 0.14 m/s. The waveforms associated with the four chains were detected when the IOP was equal to 12.75 mm Hg (1700 Pa). One significant feature of HNSWs not observed in linear waves, is that their phase velocity Vₛ is directly proportional to the force amplitude Fₘ as Vₛ~Fₘ^{1/6}, i.e. stronger pulses propagate faster. Another feature is that a solitary pulse can be engineered by tuning the mechanical and/or the geometric properties of the particles, including varying static precompression of the particles, to attain the desired wavelength, speed, and amplitude. These are seen in the arrival time and amplitude of the ISW: the dynamic force associated with the 2 mm steel spheres is about four-fold the force measured in the 1 mm steel spheres, and about two orders of magnitude higher than the 1 mm PTFE chain. Also, at a given particles' diameter, the arrival time of the ISW is proportional to the Young's modulus, and at a given material is inversely proportional to the particles' diameter. The time waveforms also reveal that regardless the size and modulus of the particles, two reflected pulses (the PSW and the SSW) are generated and their amplitude, time of flight, and duration depend on the properties of the beads. The duration of the pulse is a parameter called “contact time”: the bigger and softer the particles, the wider are the pulses. Softer beads deform more and delay the response time to the load generated by the adjacent beads. Further, the contact time T_{c} is a function of the velocity Vₛ, mass m_{b}, and contact stiffness A_{b} according to: T_{c}≈3.218 m_{b}^{2/5} V_{S}^{-1/5} A_{b}^{-2/5}.

It can be understood from this equation that a lighter and softer particle has a greater contact time, and this is visible in the numerical results. Also, some reflected pulses can consist of “twin-peaks”. This phenomenon has been observed in other solitary wave applications, including the interaction of the waves with tennis balls. The twin-peaks are not typically used or required for effective IOP measurements, but in some examples they may be recorded or used to determine characteristics of the eye or instrument. However, other reflected pulse characteristics can occur in some examples, such as waves with various frequencies and times of flight. For example, as will be discussed further below, spectrogram information can be related to intraocular pressure.

To quantify the effect of the IOP on the amplitude and time of flight of the primary reflected wave, the amplitude of the reflected wave was normalized with respect to the amplitude of the incident wave (PSW/ISW). In many examples, a monotonic dependency of wave features with respect to the pressure can be seen. Wave amplitude can be proportional to the eye pressure. When the pressure increases, the cornea becomes stiffer and less acoustic energy is converted into the cornea deformation leading to a stronger PSW. A rapid evaluation of the extreme pressures at 12 mmHg (1700 Pa) and 30 mmHg (4000 Pa) reveals that the normalized amplitude associated with the 2 mm PTFE chain increases by 20% across the interval.

A similar analysis was conducted for the TOF and overall, this feature is inversely proportional to the pressure; as the cornea becomes softer (lower IOP), the contact time between the last bead of the chain and the cornea increases, delaying the arrival of the reflected pulses. In addition, the lower the amplitude of the reflected wave the slower is its speed, increasing further the TOF of the PSW.

To quantify the sensitivity of four tested experimental chain designs with respect to the IOP variation, the numerical data were interpolated with a second degree polynomial. The equations with the highest coefficients reveal the chain that provides the highest sensitivity to the variation of the eye pressure. For example, the chain made of twenty 1-mm diameter PTFE particles was found to be the most sensitive to the IOP variation and therefore can be used in experimental validation of the example tonometers.

These models and experiments investigated numerically the effects of the intraocular pressure on the interaction between highly nonlinear solitary waves propagating along 1-dimensional chains of spherical particles and the cornea of young adults, in contact with one end of the chain. The study evaluated the feasibility of a solitary-wave based tonometer to measure the IOP. Engineering principle not yet explored in ophthalmology were applied to this biomedical problem by implementing a finite element formulation coupled to a discrete mass-spring model. It was found that the travel time and the amplitude of the waves reflected at the interface between the last particle of the chain and the cornea is affected by the internal pressure. These dependencies were quantified numerically by taking into account the fact that the stiffness of the cornea is a function of the pressure. Some disclosed apparatus and methods examples can use these principles to effect solitary wave based tonometry measurements though disclosed examples are not necessarily limited by the disclosed models and principles.

In the models and experiments associated with solitary waves, certain characteristics were ignored or simplified, such as the effect of the eyelid, and the analysis focused on a specific value of the cornea radius and thickness. The stiffness of the cornea can be understood to be a function of the pressure, loading direction, and loading rate, as well as cornea and/or eyelid stiffness and/or thickness, and the presented model can be expanded to account for a broad range of geometric and mechanical characteristics of the eyeball, including variation of selected parameters across patient groups. In some examples, selected parameters can be accounted for in measurement estimates, such as between different patients or patient subsets (age, race, sex, medical history, etc.), or as updated through additional or refined modeling.

In a clinical setting, instrument examples can be calibrated to the physiological properties of the patient's cornea, such as eyeball diameter, eyelid thickness and/or age, and corneal thickness and modulus, and corneal radius, thickness, and modulus can be quantified to determine how physiological parameters affect solitary wave features and suitable parameter ranges for solitary-wave based tonometry applicability. In some examples, acquired patient-specific information can be used by the solitary wave-based tonometer (e.g., input by a user, inferred through solitary wave detection, or determined from other detection) to automatically or manually adjust device settings, including change solitary wave characteristics. As will discussed further below, selected examples can leverage accurate measurements of corneal thickness to improve IOP measurement accuracy.

### EXAMPLE EMBODIMENTS

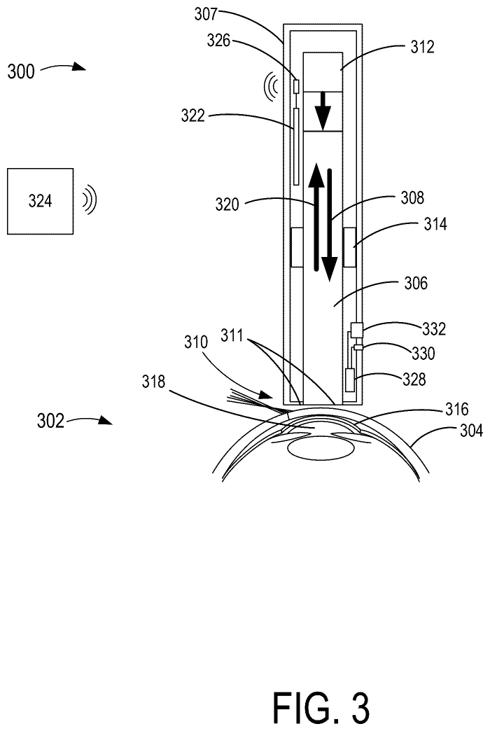

FIG. 3 is an example tonometer 300 positioned to measure an IOP of an eye 302. The eye 302 and tonometer 300 are shown schematically and cross-sectionally to illustrate basic operation. An eyelid 304 of the eye 302 may be closed during the measurement, advantageously allowing for easier positioning and self-application of the tonometer 300 to perform the measurement. The tonometer 300 can include a stress wave carrier 306 retained in a housing 307. The housing 307 can be ergonomically constructed so that the tonometer 300 may be held and applied by a user, such as the person whose eye 302 is being measured for IOP. The stress wave carrier 306 can be situated to propagate an incident mechanical stress wave 308 to an end 310 of the tonometer 300 to contact the eyelid (or cornea of the eye in some examples).

In representative examples, the stress wave carrier 306 is a particle array, such as a string of particles adjacently arranged end to end to contact each other and transmit the incidence stress wave 308 from one particle to the next. In many examples, the particles can be spherical, though other shapes may be used. Spherical particles can have a diameter on the order of a few mm, e.g., 0.5 mm, 1 mm, 2 mm, 4 mm, 8 mm, etc. In many examples, particles are made of a singular material, such as metal, plastic, ceramic, etc. In some examples, other materials or material configurations may be used for the stress wave carrier 306, such as a monolithic structure. The end 310 can include a retainer element 311 to retain the contents of the stress wave carrier 306 in the housing 307, such as with a flexible membrane and/or ridged surface, etc. In many examples, the mechanical stress wave 308 can be in the form of a solitary wave, such as a highly nonlinear solitary wave (HNSW).

A striking mechanism 312 can be situated in the tonometer 300 to cause the mechanical stress wave 308 to propagate. Various striker mechanisms may be used, including but not limited to compressed springs, dropped objects (such as another particle of the particle array held above the particle array to fall on the particle array), piezoelectric transducers, etc. After the striking of the wave carrier 306, the stress wave 308 propagates through the wave carrier 306 and stress wave characteristics are detected by a detector 314. The incident stress wave 308 interacts with the eye 302, e.g., propagating through the eyelid 304 and cornea 316 to the anterior chamber 318. One or more return mechanical stress waves 320 propagate back along the particle array 306 and their characteristics are detected by the detector 314. In representative examples, time-of-flight is measured for primary and/or second solitary waves. In further examples, more detailed wave characteristics are measured allowing further analysis and extraction of additional information, such as both time and frequency domain data.

Examples of the detector 314 can include a magnet and coil of wire configured to exhibit an electrical variation in response to the stress wave 308 via the magnetostrictive effect. The wave carrier 306 can be locally magnetized with the magnet and the propagating stress waves (such as waves 308, 320) can induce a current in the coil via the magnetostrictive effect. The current can be sensed and/or converted to a voltage detectable by the controller 322. Other detectors may be used as well, with further examples discussed elsewhere herein.

The tonometer 300 can include a controller 322, typically including a printed circuit board and configured with one or more processors and memories that can be configured to carry out various IOP measurement functions. For example, the controller 322 can be coupled to the detector 314 to receive detection data and store, analyze, and/or transmit the data or analyzed data to a remote device (e.g., wired or wirelessly). The controller 322 can also be coupled to the striking mechanism 312 to control an initiation and timing of the stress wave 308 and to track detection events in relation to strike initiation. Where analysis is to be performed locally, e.g., in the tonometer 300, the controller 322 can be configured to analyze the detected data to estimate IOP. Alternatively, analysis can be performed remotely, e.g., with a local wired or wirelessly connected device 324 (such as a handheld smartphone or tablet) or at a remote location. Wireless connections can be made with a wireless transceiver 326 coupled to or part of the controller 322 (e.g., via Wi-Fi, Bluetooth, or near field communication protocols).

IOP estimates and analyses can be obtained in various ways in the device 300 with the controller 322 or remotely. In some examples, estimates can be obtained by comparing primary and/or secondary solitary return wave time of flight to a stored correlation between IOP and time of flight. Some examples can use other wave characteristics and related correlations to IOP, such as frequency content, period, number of oscillations, or other wave signatures. In further examples, detected data and/or wave characteristics can be processed through a machine learning tool, such as an artificial neural network, with the machine learning tool producing estimates based on the wave characteristics, such as time domain and/or frequency domain characteristics. In some examples, the machine learning tool can be trained on wave characteristics and ground truth IOPs obtained from various eyes or simulated eyes. In some examples, the machine learning tool can be provided with one or more additional inputs, such as a corneal thickness measurement obtained separately using another device, such as with a pachymeter, confocal microscope, optical coherence tomography (OCT) system, etc. Other additional inputs can include eyelid characteristics (where the tonometer is positioned over the eyelid) like eyelid thickness. In some examples, wave characteristics that are inputs to the machine learning model can correspond to spectrograms. Spectrograms generally include time and frequency domain data for a signal along with amplitude. Spectrogram data can be represented visually in two-dimensions, typically with a time axis, a frequency axis, and a color, intensity, or other weighting scheme to represent signal amplitude at a particular frequency and time. Herein, spectrograms can refer to visual representations along with corresponding time, frequency, and amplitude data.

In some examples, the tonometer 300 can include an inclinometer 328, e.g., in the form of a gyroscope and/or accelerometer, that can be coupled to the controller 322. The inclinometer 328 can be configured to detect alignment characteristics of tonometer 300 during use of the device. For example, the inclinometer 328 can detect a positioning of the tonometer 300, and more particularly the wave carrier 306, in relation to the gravitational field of the Earth. This detection can be used to ensure the wave carrier 306 is in a suitable orientation to transmit the incident wave 308 and receive the return wave 320 during a measurement. For example, the inclinometer 328 can be used to provide feedback to the user performing the measurement regarding how the tonometer 300 is spatially situated in order to improve IOP prediction accuracy.

In many examples, a suitable orientation is vertical, i.e., aligned with Earth's gravitational field. The suitability of the orientation can correspond to a range of angles with respect to the Earth's gravitational field, e.g., ±20°, ±10°, ±5°, ±1°, etc. In some examples, this orientation can be associated with a predetermined strike force (e.g., maximum) imparted to the wave carrier 306 by the striker mechanism 312. In some examples, the controller 322 can be configured to inhibit measurement acquisition outside of a suitable orientation, e.g., by disengaging the striker mechanism 312, not recording or detecting wave data, and/or indicating unsuitability for measurement on a display (locally on the tonometer 300 or remotely). Thus, the tonometer 300 can be configured to improve measurement accuracy by verifying or coordinating measurement in a supine position. In some examples, orientation data provided by the inclinometer 328 can be used as an input to a machine learning tool, such as example machine learning tools and processes described herein.

In some examples, a light 330 can be situated to emit a visual indication responsive to a position detection by the inclinometer 300. For example, the light 330 can emit light when the position is suitable (constant, blinking, etc.) or it can emit light when the position is not suitable. In some examples, the tonometer 300 can include a speaker 332 or other audible device configured to produce a sound that can be heard by a user and that can thereby provide an indication of the suitability of the position detected by the inclinometer 328. For example, the speaker 332 can produce a sound when the positioning is suitable, or it can produce a sound when the position is not suitable.

In some examples, the positioning detection provided by the inclinometer 328 can be used additionally or alternatively to detect undesirable movement of the tonometer 300 by the user that has readied the tonometer 300 for a measurement. For example, after the end 310 of the tonometer 300 is positioned in a suitable orientation and to be in contact with the eyelid 304 of the eye 302, the inclinometer 310 can detect excessive motion of the tonometer with respect to the eyelid 304, such as an excessive motion or a movement indicative of an undesirable additional pressure applied to the eye 302 that could reduce an IOP measurement accuracy.

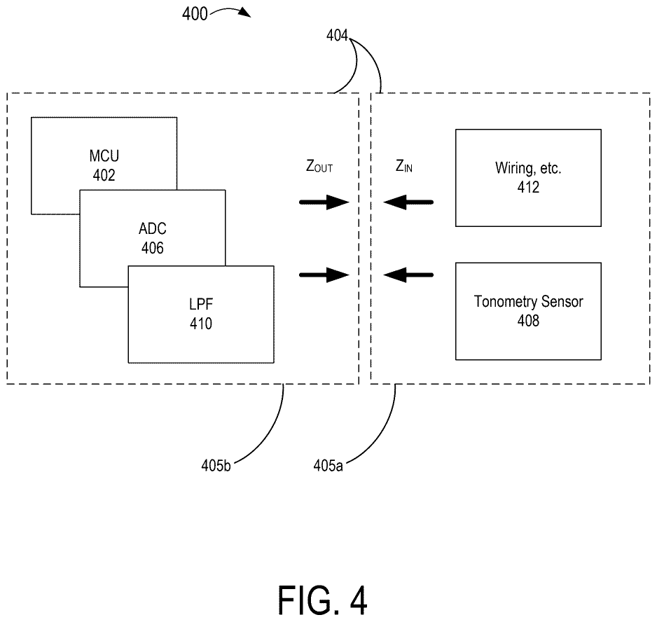

FIG. 4 is an example controller and sensor system 400 that can be used in any of the tonometer device examples and methods described herein. The system 400 includes a microcontroller unit (MCU) 402 which typically includes one or more processors and memories configured to execute processor-executable instructions for carrying out various system functions like control, detection, and I/O. The MCU 402 can be coupled to part of a larger electrical circuit 404 that can include a circuit of various components 405a coupled to an MCU circuit 405b of the MCU 402 to carry out such system functions. The MCU circuit 405b can include one or more analog to digital converters (ADC) 406, and one or more low pass filters 410 or other filters. The various components 405a can include a tonometry sensor(s) 408 and miscellaneous electrical components 412 like wiring, resistors, capacitors, diodes, light emitting elements, transistors, antennas, etc.

The coupling of components within the sensor system 400 in the circuit 404 can be viewed as an equivalent circuit in which the MCU circuit 405b can have a characteristic source impedance Zour seen by the circuit of the various components 405a, and the circuit of the various components 405a can have a characteristic load impedance ZIN seen by the MCU circuit 405b. In many disclosed examples, the source and load impedances are matched closely, e.g., within ±1%, ±5%, ±10%, ±25%, etc. In many examples, the detection signals can have a very low signal power and be highly sensitive to electrical reflections caused or influenced by the various components of the system 400, including mechanical components such as wave carrier particles.

Such reflections have the potential to destroy or decrease the quality of the detection signal. By closely matching the impedances, electrical reflections of the detection signal can be minimized. In some examples, various components can be selected to achieve impedance matching, e.g., using an ADC with a selected impedance that causes the load impedance ZIN to align with the source impedance Z_{OUT}. Thus, in representative examples in which a tonometer wave carrier is arranged to propagate one or more stress waves to an eye, an electrical circuit like circuit 404 can be coupled to a sensor detecting the incident and return stress waves from the wave carrier, and an MCU (like MCU 402) can be coupled to the sensor to detect an electrical signal from the sensor. The electrical circuit can be impedance matched to reduce electrical reflections that deteriorate the electrical signal received by the MCU 402. In some examples, the low pass filter 410 can be situated to reject noise associated with the tonometry detection signal. In some examples, the filter 410 can provide a cutoff frequency of 0.3 MHz, 0.5 MHz, 1 MHz, 10 MHz, etc., which can be configured to remove excess high frequency noise. In some examples, the detection signal can be configured to peak near 30 kHz.

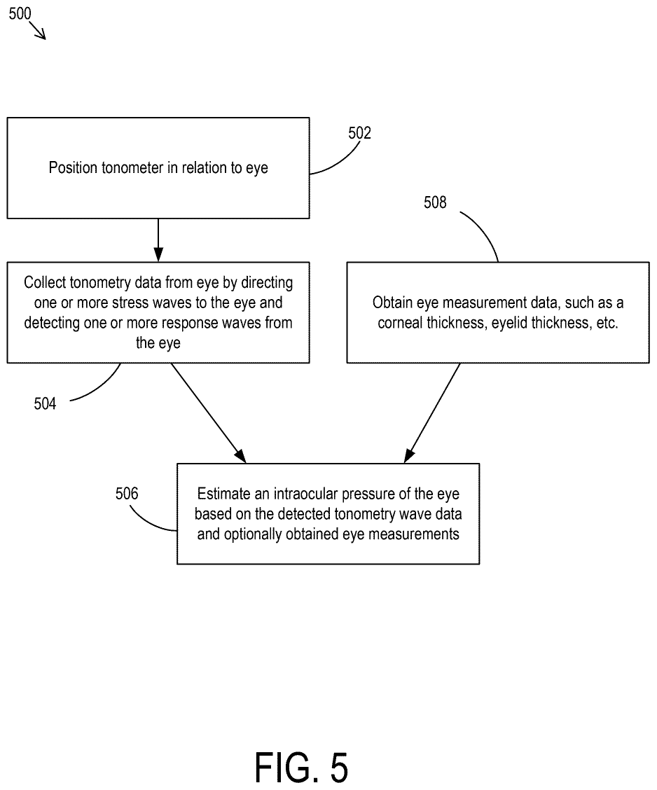

FIG. 5 is an example method 500 of estimating an intraocular pressure. At 502, a tonometer is positioned in relation to an eye to be measured. In many examples, the tonometer can be handheld and positionable by a user, e.g., through self-administration or by another person. In many examples, the tonometer can be positionable in a preferred or optimized orientation associated with tonometer operation and sensing mechanisms, such as vertical. In further examples, other orientations may be preferred (such as horizontal) or orientation may not be a factor in operation and sensing (i.e., it may be performed in any position). In many examples, the positioning of the tonometer at 502 can include bringing a terminal or sensory end of the tonometer into contact with a closed eyelid. The positioning can be easier and less intrusive than positioning required by other existing tonometers, such as those requiring direct contact with the cornea. In some examples, the tonometer can include a gyroscope and/or accelerometer (which can be referred to as an inclinometer) that can be used to detect an orientation and/or movement of the tonometer in relation to the eye. Various examples can use the detected orientation and/or movement to prevent one or more detection steps unless a suitable orientation for the device is achieved, provide an indication to the user that a suitable orientation is achieved or is not achieved, prevent one or more detection steps unless a suitable pressure condition is achieved (e.g., preventing measurement where an excessive pressure is applied to the eyelid), and/or to adjust IOP estimates based on the detected orientation and/or movement.

At 504, tonometry data relating to intraocular pressure is collected from the eye by directing one or more stress waves, such as incident solitary waves, to the eye and detecting one or more response stress waves, such as primary and/or secondary reflected solitary waves, that propagate back to the tonometer from the eye. At 506, the tonometer then estimates, within the tonometer itself or remotely, an intraocular pressure of the eye based on the detected tonometry stress wave data. In some examples, separate eye measurement data, such as a corneal thickness, eyelid thickness, etc., can be obtained at 508 and provided for the estimation process. For example, some estimations can be performed with a machine learning tool, such as an artificial neural network, that is trained on a relation between detected stress wave characteristics and intraocular pressures for various eyes. In some of such examples, the neural network can include training with corneal thickness data and/or eyelid thickness data, such that the IOP measurement effects of corneal thickness and/or eyelid thickness can be factored into the model output where a corneal thickness and/or eyelid thickness measurements are provided as inputs to the model alongside detected stress wave data.

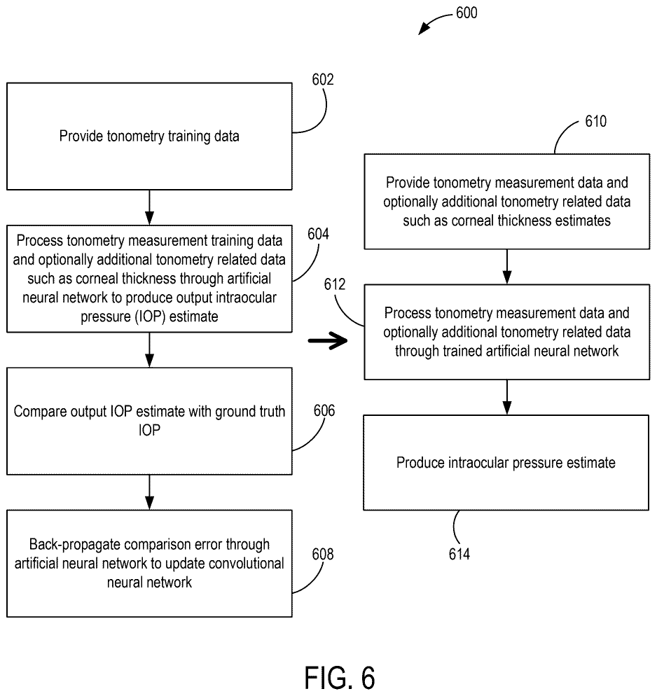

FIG. 6 is an example IOP estimation machine learning framework 600 using an artificial neural network, such as a convolutional neural network. The framework 600 can include a training portion 602-608 that can be configured to refine parameters of the artificial neural network, e.g., to improve output accuracy as additional training data sets are processed. At 602, a set of tonometry training data is provided to the artificial neural network. Training data typically includes a set of tonometry measurement data for various eyes having a known, ground truth intraocular pressure (e.g., corresponding to measurements obtained through a separate tonometer) and optionally other characteristics like corneal thickness or eyelid thickness. At 604, the training data is processed through the artificial neural network to produce an intraocular pressure estimate as an output. At 606, the output intraocular pressure estimate is compared to the ground truth intraocular pressure associated with the training data to determine a comparison error. At 608, the artificial neural network is updated, e.g., by updating activations of one or more network layers by back-propagating (e.g., through gradient descent) the comparison error through the artificial neural network.

A measurement phase 610-614 can be used on eyes to be measured, after the artificial neural network is sufficiently trained. At 610, tonometry data can be provided as an input to the trained artificial neural network. Tonometry data can be collected for an eye by directing one or more stress waves to the eye and detecting stress wave response characteristics. In some examples, additional tonometry data can be provided as an input to the artificial neural network, such as a corneal and/or eyelid thickness estimates or measurements of the eye. At 612, the data are processed through the trained artificial neural network, and at 614 an intraocular pressure estimate output is produced based on the input tonometry data.

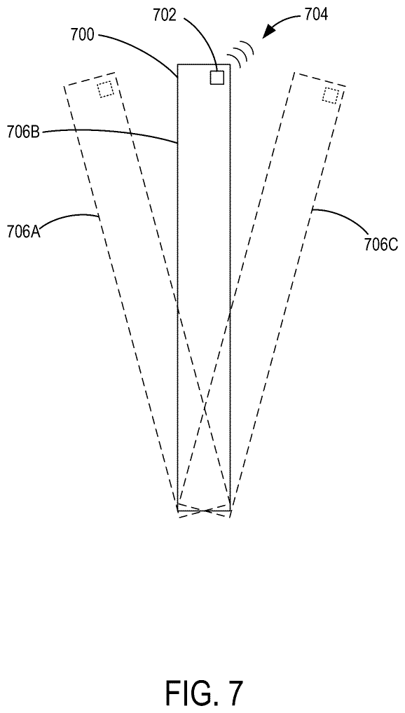

FIG. 7 is an example tonometer 700 that can be used to measure an intraocular pressure of an eye, in accordance with various examples described herein. The tonometer 700 includes an internal inclinometer 702 that include an accelerometer and/or gyroscope to detect an orientation of the tonometer 700 in relation to Earth's gravitational field. The tonometer 700 can include a visual or audio source 702 that can be coupled to the inclinometer 702 (e.g., directly or via an intermediate controlling unit) to produce a visual or audible indication 704 in response to the inclinometer detecting a selected orientation of the inclinometer. For example, as shown in FIG. 7 with the vertical direction of the figure generally aligned with the Earth's gravitational field, the tonometer 700 can be moved to different angled positions 706A-706C. During use of the tonometer 700, the inclinometer 702 can be configured to produce the indication 704 only when the orientation is suitable for obtaining an accurate measurement, thereby indicating to the user that a measurement can proceed. In further examples, an indication can be provided when the orientation is not suitable, or a range of indications can be provided based on orientation, such as a change in pitch and/or rhythm, etc.

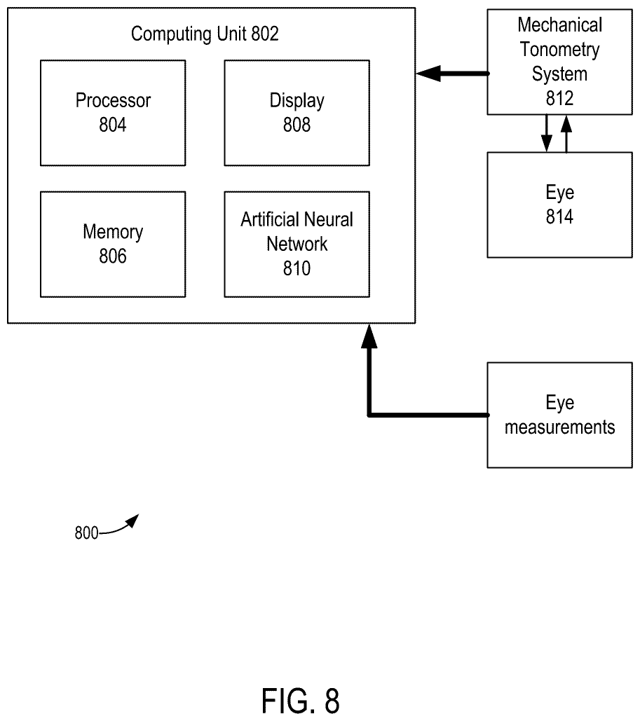

FIG. 8 is an example tonometry system 800 can be part of various devices described herein, correspond to components of various devices described herein, and/or configured to implement various methods described herein. The system 800 includes a computing unit 802 that can include one or more processors 804, memory 806, a display 808, and various software routines, such as a trained artificial neural network 810. The computing unit can be coupled to a mechanical tonometry system 812 that is coupled to an eye 814 to carry out intraocular pressure measurements of the eye 814. The trained artificial neural network 810 can receive tonometry measurement data from the mechanical tonometry system 812, such as detected stress wave characteristics like a signal amplitude that varies over time. In some examples, the processor 804 can be configured to convert the detected stress wave characteristics to spectrogram data, e.g., by digitally processing the digitally sampled detected stress wave signal with a fast Fourier transform (FFT) or short-time Fourier transform (STFT). Separate eye measurements 816, such as corneal thickness measurements, eyelid thickness measurements, or other information relating to the eye 814, can be coupled to the computing unit 802. For example, a user can enter separate measurement data through a user interface, such as software application on a handheld device. The separate eye measurements 816 can be used to enhance the estimation accuracy provided by the artificial neural network 810 by providing additional data inputs that are associated with IOP measurement correlations.

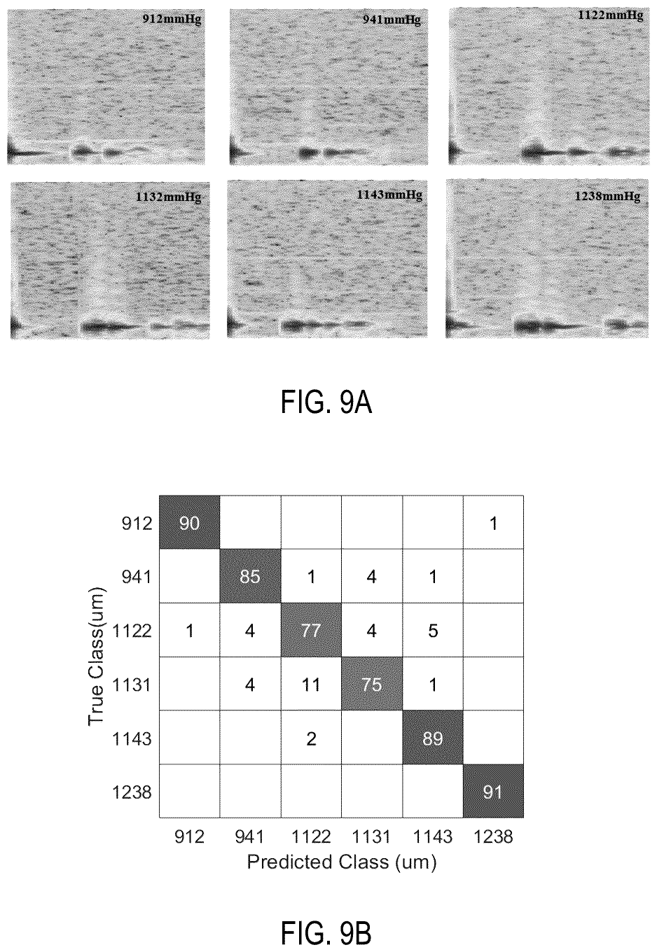

FIG. 9A shows six spectrograms obtained from tonometer detection signals in a test apparatus. The test apparatus included a pressure vessel inflate to a predetermined pressure, simulating an intraocular pressure. A corneal simulation layer of thicknesses 912 μm, 941 μm, 1122 μm, 1131 μm, 1143 μm, and 1238 μm covered the pressure vessel for different test runs, with the corresponding spectrograms being shown in the top left, top middle, top right, bottom left, bottom middle, and bottom right, respectively, in FIG. 9A. Results of processing the spectrograms through a trained machine learning model to predict the corneal simulation layer thickness are shown in FIG. 9B. As shown, a relatively high test accuracy of approximately 93% was achieved.

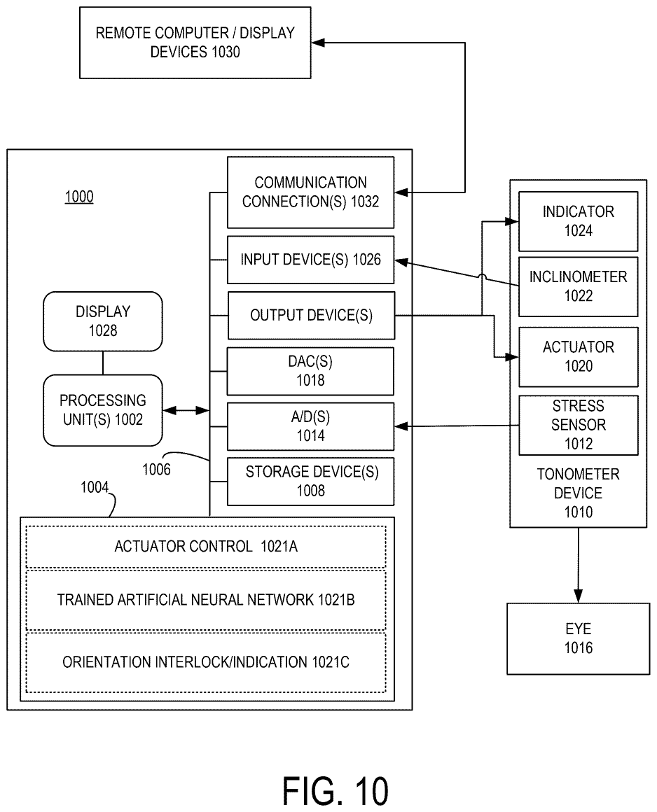

FIG. 10 shows another example implementing tonometry detection on a hardware platform, such as a computing device 1000. In general, the following discussion provides a brief, general description of an exemplary computing environment in which the disclosed stress-wave based tonometry detection and IOP estimation techniques may be implemented. Although not required, the disclosed technology is described in the general context of computer-executable instructions, such as program modules, being executed by a computing unit, dedicated processor, multiple processors, or other digital processing system or programmable logic device. Generally, program modules include routines, programs, objects, components, data structures, etc., that perform particular tasks or implement particular abstract data types. Moreover, the disclosed technology may be implemented with other computer system configurations, including hand-held or mobile devices, personal computers (PCs), multiprocessor systems, microprocessor-based or programmable consumer electronics, network PCs, minicomputers, mainframe computers, dedicated processors, MCUs, PLCs, ASICs, FPGAs, CPLDs, systems on a chip, and the like. The disclosed technology may also be practiced in distributed computing environments where tasks are performed by remote processing devices that are linked through a communications network. In a distributed computing environment, program modules may be located in both local and remote memory storage devices. For example, processing (including function generation, waveform digitization, machine learning tool processing (such as with a ANN)) can be distributed between local and remote devices. In some examples, intensive processing can be dedicated to remote computers or mobile devices.

With reference to FIG. 10, an exemplary system for implementing the disclosed technology includes the computing device 1000 that includes one or more processing units 1002, a memory 1004, and a system bus 1006 coupling various system components, including the system memory 1004, to the one or more processing units 1002. The system bus 1006 may be any of several types of bus structures including a memory bus or memory controller, a peripheral bus, and a local bus using any of a variety of bus architectures. The memory 1104 can include various types, including volatile memory (e.g., registers, cache, RAM), non-volatile memory (e.g., ROM, EEPROM, flash memory, etc.), or a combination of volatile and non-volatile memory. The memory 1004 is generally accessible by the processing unit 1002 and can store software in the form computer-executable instructions that can be executed by the one or more processing units 1002 coupled to the memory 1004. In some examples, processing units can be configured based on RISC or CISC architectures, and can include one or more general purpose central processing units, application specific integrated circuits, graphics or co-processing units or other processors. In some examples, multiple core groupings of computing components can be distributed among system modules, and various modules of software can be implemented separately.

The computing device 1000 can further include one or more storage devices 1008 such as a hard disk drive, flash drive, etc., which can be connected to the system bus 1006 by a storage communications interface. The drives and their associated computer-readable media provide nonvolatile storage of computer-readable instructions, data structures, program modules, and other data for the computing device 1000. Other types of non-transitory computer-readable media which can store data that is accessible by a computing device may also be used in the exemplary computing environment. The storage 1008 can be removable or non-removable and can be used to store information in a non-transitory way and which can be accessed within the computing environment.

The computing device 1000 can be coupled through one or more analog to digital convertors (A/Ds) 1014 to a stress wave sensor 1012 housed in the computing device 1000 (forming a tonometer unit) or in a separate tonometer device 1010. Thus, in some examples, the computing device 1000 (or selected parts of the computing device 1000) can be integrated into a tonometer unit that can couple to an eye 1016. In some examples, the computing device 1000 with stress wave sensor 1012 can comprise application specific hardware/software, such as the tonometer unit, specifically configured for detection of solitary waves and estimation of intraocular pressure based on characteristics of the detected solitary waves. During operation, the stress wave sensor 1012 detects stress wave characteristics (e.g., stress wave amplitude that varies over time, such as for incident waves, primary and/or secondary reflected solitary waves, and other waves) associated with propagation and reflection of stress waves along a wave carrier (such as a chain of particles) to and from an eye 1016. The stress wave sensor 1012 detects the passing stress waves and produces a stress wave signal, typically in the form an electrical current that varies over time based on the detected stress wave characteristics. The stress wave sensor 1012 sends the stress wave signal to the computing device 1000 for signal analysis and production of an IOP estimate for the eye 1016. The computing device 1000 can include digital to analog converters (DACs) 1018 coupled to the bus 1006, e.g., for control of external analog devices, such as an actuator 1020 used to produce the incident stress wave that propagates along the wave carrier. Various actuators may be used, such as a solenoid-controlled striker, piezoelectric transducer, etc. In many examples, the equivalent circuit coupling between the processing unit 1002, the A/D 1014, and other components (such as an input filter) is impedance matched with respect to wiring, the sensor 1012, and other components, to reduce undesirable loss or attenuation of the detected stress wave signal.

The software, e.g., stored in the memory 1004 at 1021A, can automate the measurement of IOP for a user by controlling the actuator to generate a suitable stress wave in the wave carrier. Example functions that produce stress waves can include square waves, sinusoidal waves, simple pulses, variable pulses, etc. The memory at can further include digitization routines that can be used to digitize the detected stress wave signal. In further examples, the waveform digitization can be performed in hardware and/or in a device separate from the computing device 1000.

The memory at 1021B can include a trained artificial neural network (such as a CNN) that can be configured to receive stress wave signal data, such as data representing amplitude with respect to time, time-of-flight, frequency spectra, and/or spectrograms, as an input, and IOP as an output. Other inputs can include, e.g., corneal thickness, eyelid thickness, or other characteristics. Inputs can be used to improve the IOP estimation accuracy. In some examples, memory can include a mapping between stress wave characteristics and IOP (e.g., with a look-up table) to produce an estimate of an IOP of the eye 1016. For example, alternative estimates can be produced by comparing characteristics of the digitized waveform, such as a monotonic dependence between IOP and amplitude and time-of-flight (ToF) of one or more of the reflected stress waves (including primary and secondary waves or multiple wave samples) or other waveform characteristics, such as amplitudes of incident, primary, and/or secondary stress waves, time of flight of primary and/or secondary stress waves, a width at half amplitude of each of the three waves, and any declination in terms of their ratios or product, such as the ratio of the amplitude of the PSW to the amplitude of the ISW or the product of the two amplitudes.

In some examples, the software, e.g., stored in the memory 1021C, can automate control of the measurement based on certain orientation criteria. For example, an inclinometer 1022 can be coupled to the tonometer device 1010 to sense an orientation of the device 1010 with respect to Earth's gravitational field. The software can be configured to prevent or allow measurement based on the detected orientation. In some examples, an indicator 1024 can be configured to provide an audio and/or visual indication responsive to the detected orientation and/or suitability of orientation for measurement.

In addition to the above, a number of program modules (or data) may be stored in the storage devices 1008 including an operating system, one or more application programs, other program modules, and program data. A user may enter commands and information into the computing device 1000 through one or more input devices 1026 such as a keyboard, a pointing device such as a mouse, or control buttons to initiate or control a tonometry test or to display an IOP estimate. The inclinometer 1022 can correspond to one of the input devices. Other input devices may include a digital camera, microphone, satellite dish, scanner, display, or the like. These and other input devices are often connected to the one or more processing units 1002 through a serial port interface that is coupled to the system bus 1006, but may be connected by other interfaces such as a parallel port or universal serial bus (USB), or integrated wiring. A display 1028 such as an LCD display, monitor, or other type of display device can also be connected to the system bus 1006 via an interface, such as a video adapter. Some or all data and instructions can be communicated with a remote computer 1030 through communication connections 1032 (e.g., wired, wireless, etc.) if desired. In some examples, the remote devices 1030 can include one or more mobile devices or other computing devices that can be used to provide the majority of signal generation, processing, and/or IOP estimation, preferably with the computing device 1000 having pared down functionality sufficient to provide integration of the computing device 1000 with the stress sensor 1012 as a tonometer unit so that the tonometer unit can be hand-held by a user to self-administer the tonometer device to the user's eye. In some examples where the stress sensor 1012 is part of the tonometer device 1010 and separate from the computing device 1000, the computing device 1000 can be a mobile device, such as a smartphone or hand-held computing unit.

### Additional Experiments

In additional experiments, the effects of corneal thickness on intraocular pressure were closely examined. Tonometers should be able to measure IOP and preferably account for the effects of corneal anatomy. The sensitivity of stress wave characteristics in relation to IOP and corneal thickness were investigated by testing with polydimethylsiloxane (PDMS) corneas, named cornea eyeball phantoms (CEPs). Five CEPs were fabricated with central corneal thicknesses (CCT) between 492 μm to 642 μm. To gauge the mechanical properties of the fabricated corneas, and their similarity to human corneas, compressive tests were performed resulting in an average Young's modulus of 453 kPa. The CEPs underwent controlled pressure tests where IOP was swept from 12 to 26 mmHg while recording HNSW waveforms during each step. The collected waveforms were then used to infer the device's capabilities to produce distinct signals that can be correlated with IOP. Due to its salience, the time between peaks in a HNSW was measured as it produces insightful information that correlates to both IOP and CCT. To further explore the ability to distinguish differences in CCT, the HNSW results were analyzed in the time-frequency domain via the short time Fourier transform (STFT) and used as input to a machine learning classification algorithm. This method of analysis resulted in the thickness of the CEP being predicted with an 89.15% accuracy. The results indicated that disclosed tonometers can be used to accurately identify the IOPs of different eyes having different CCTs.

In the experiments, the five PDMS CEPs were fabricated from the same batch at a 20:1 ratio, resulting in an average Young's modulus of 453 kPa and CCT ranging from 492 μm to 643 μm. Each CEP was pressurized from 12 mmHg to 26 mmHg in 1 mmHg increments. Thousands of solitary wave measurements were taken during the experiments using a lab-built transducer. A first investigation involved the extent to which HNSWs from the same CEP would produce distinct, time-domain features that correspond to IOP pressure values between 12-26 mmHg. These trends in PDMS CEPs were compared to that of an animal model (a lamb) with a similar CCT. A second investigation involved whether the HNSWs would also produce features impacted by CCT. The tonometer's ability to distinguish changes in the HNSW signal mapped to the CCT of each PDMS cornea was examined.

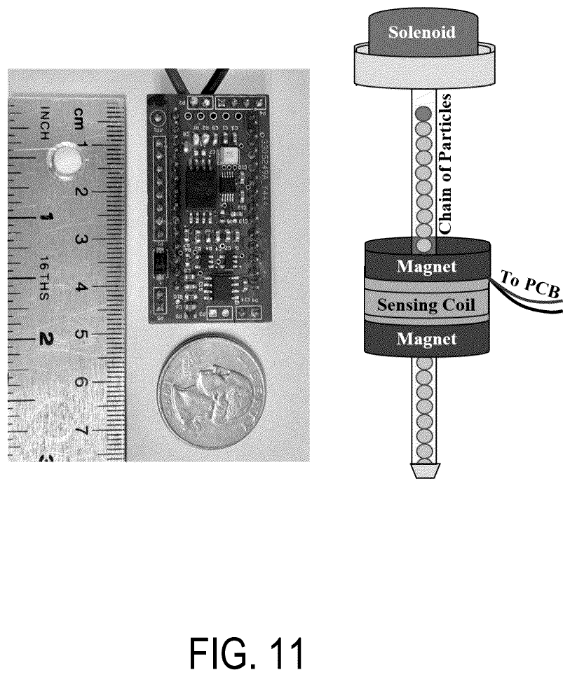

Tonometers used in the experiments were made in accordance with disclosed examples herein. As shown in FIG. 11, a transducer array used for measuring HNSWs in this design is formed by a linear array of 25 particles. Atop the array was a commercially available solenoid. The gap between the solenoid and the top-most spherical particle, hereinafter referred to as the striker, was the diameter of one particle: 2.38 mm. ISWs were generated mechanically by lifting and releasing the striker. The waves were sensed via a 10 mm long coil made of 36 AWG electromagnetic wire. The coil allows the wave to be sensed by using the inverse magnetostrictive effect. This effect operates via a pulse that propagates through a ferromagnetic material and also modulates a strain in the material. This modulation in turn modifies an existing, permanent magnetic field and ultimately creates current in the coil. The coil is composed of 1500 turns with a resistance of 84Ω. The array is housed in a 3D printed frame made of a clear resin. As shown in FIG. 11, the printed circuit board (PCB) used for collecting HNSWs measures 48 mm×25 mm.

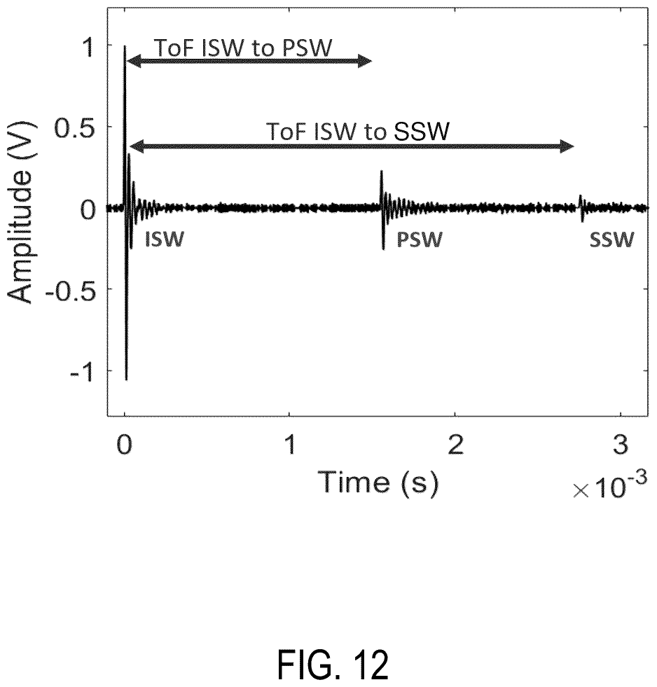

The striker and the four particles surrounded by the sensing coil are made of ferromagnetic materials, whereas the remaining particles are made of stainless steel. After the ISW reaches the opposite end of the chain, the area in contact with the test specimen, it reflects off the test specimen and travels back through the chain of particles creating primary solitary waves (PSWs) and secondary solitary waves (SSW). The form of a typical HNSW is shown in FIG. 12. One of the most notable features of an HNSW is the time-of-flight (ToF), which can be measured as the time between either the ISW and PSW or ISW and SSW. As shown, a HNSW waveform is overlaid with features used in this work. The features include the ToF, ratio of PSW to ISW, and the power amplitude of the frequency composition.

Circuitry is impedance matched along the path from the sensor to the analog-to-digital converter (ADC) input to ensure that the processing and transmitting the information carrying signals is performed with reduced loss or attenuation. This can be particularly challenging because the overall impedance can involve a complex combination of the particle chain and sensing coil. Without proper matching, unwanted electrical reflections will be present in the return signal and can obfuscate the actual HNSW. Proper matching was achieved by converting all differential components to single-ended throughout the entire signal pathway up to the data collection performed by the MCU. By carefully designing the circuit under these constraints, unwanted oscillations are eliminated from the sampled HNSWs.

The five artificial corneas used in the experiments were fabricated using PDMS mixture Sylgard 184, a material that has been shown to be a proxy for human corneas. The mixture contains a silicon base and curing agent. A typical curing ratio of PDMS is 10:1 resulting in a Young's modulus equal to ~2.05 MPa, which is too high for glaucomatous patients. For example, clinical observations show that in early onset glaucomatous patients, the average Young's modulus is 328 kP, increases to 392 kPa at 60 years old, and becomes 488 kPa at 90 years old. The Young modulus of the final specimen can be altered by changing the base to agent ratio, and by modifying the bake time and temperature. In the experiments, a mixing ratio of 20:1 was used, as the literature shows that PDMS made with this ratio results in a Young's modulus between 340 kPa to 500 kPa when the bake temperature is varied from 60° C. to 100° C. The CEPs were prepared using a standard degassing procedure with a bake time of 2 hours at 80° C.

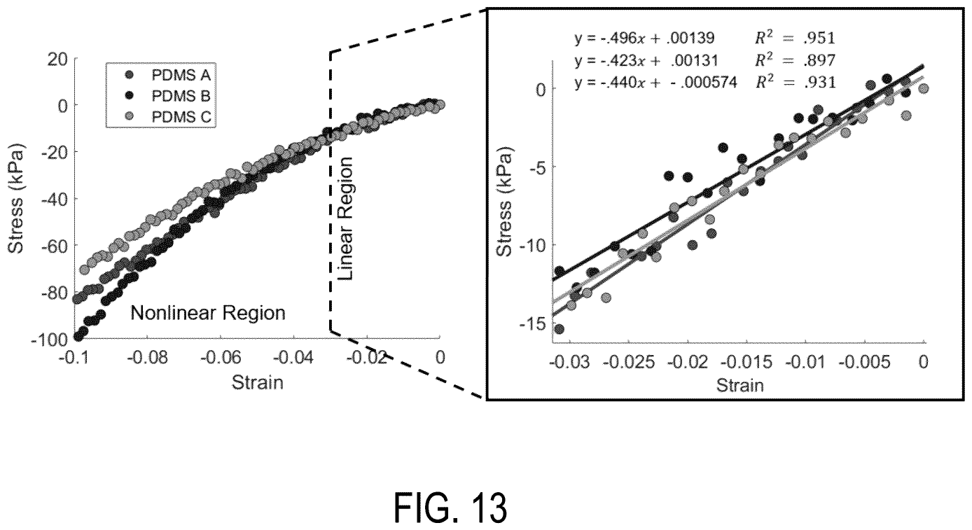

The mechanical properties of the CEP test specimens were estimated empirically by manufacturing three test coupons using the same ratio, bake time, and temperature. Each coupon was 18 mm×18 mm, with two coupons having a thickness of 3.44 mm, and the third having a thickness of 3.23 mm. The three samples were tested in compression using a fatigue testing station (Instron 8874). A displacement-controlled compression test was conducted, subjecting samples to strains ranging from 0% to 10% throughout their thickness. Each sample was positioned between two flat plates to ensure uniform application of force across its entire surface during the experiment. The compression test results were a superior model of PDMS performance and were deemed more accurate compared to a tension test. The results of the three experiments in terms of stress vs strain are shown in FIG. 13. The slope of the linear region of the stress-strain plot was used to estimate the Young's modulus of each coupon. It was estimated that the average Young's modulus of the test samples was 452 kPa, which aligns well with the clinical values found in glaucomatous patients.

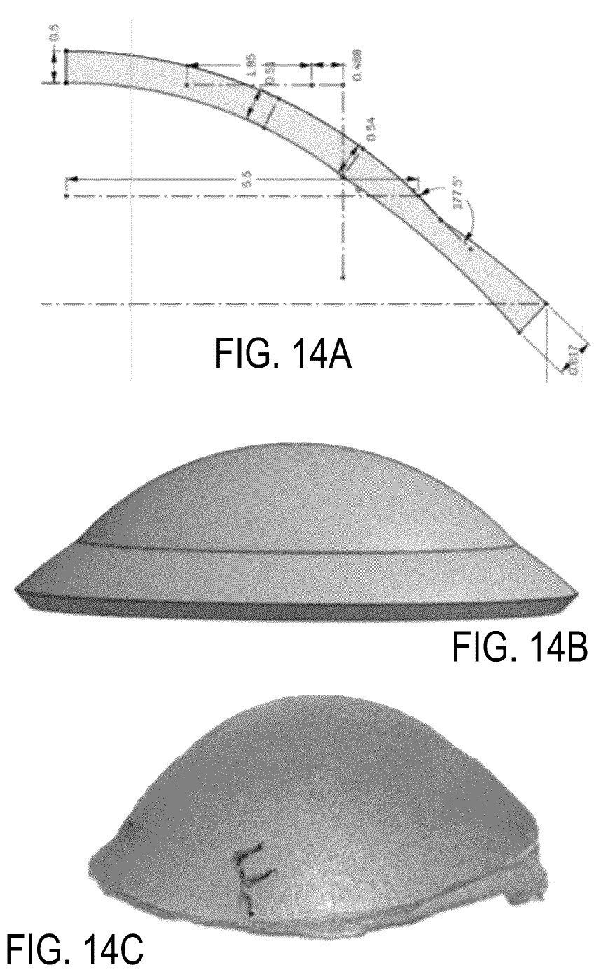

The test specimens were poured into a mold for baking, with each mold being printed using clear resin. The CEP had a radius of curvature of 7.8 mm and a corneoscleral junction of 177.5°. The corneal length and width used in the experiments differed from a typical human cornea, e.g., the model cornea was a complete circle with a 11 mm diameter compared to the typical oblong shaped human cornea having dimensions 11-12 mm horizontally and 9-11 mm vertically. The thickness measurements from the center of the cornea, where the CCT is measured, towards the corneal junction were obtained from the dimensions of a human cornea. FIGS. 14A-14C show the cross section of the model cornea, its 3D rendering, and one of the test specimens. The mold used to fabricate the test specimens targeted thicknesses between 500-600 μm, which accounts for a regular biological range of the eye thickness in glaucoma and healthy patients. The actual thickness of the CEPs were 492 μm, 498 μm, 555 μm, 560 μm, 634 μm. The measurements were taken with an optical coherence tomography (OCT) device while the CEP was clamped in place within an artificial anterior chamber.

The CEPs had a common Young's modulus for testing. Each cornea was fixed on its own individual anterior chamber and imaged with the OCT before pressure testing. Each CEP was connected to the test setup where the pressure is inflated from 12 mmHg to 26 mmHg in 1 mmHg increments. One port of the anterior chamber was connected to a water column whereas the second port was connected to a pressure sensor, which monitored the pressure of the water column and interacted with the device and a motor. First and second pressure ramps were performed sequentially and automatically, with minimum and maximum pressures and the pressure increment selected for each. The transducer is connected to the PCB through the solenoid and the sensing coil. The PCB controlled when the solenoid initiated the HNSW and collected data from the coil at 875 kHz. The PCB additionally interfaces with a motor controller to control the motor working in a feedback loop with the pressure sensor to maintain the pressure at each increment long enough to generate 15 striker impacts. After each HNSW, the PCB could save the data by sending it wirelessly or through serial communication to a computer. During testing, the transducer sits atop the anterior chamber with the bottom particle as the only point of contact with the CEP. Given that 15 measurements were taken at each of the 15 different pressures, a total of 225 time waveforms were collected at each ramp, resulting in 2,250 waveforms being collected for two pressure loading ramps against each of the five specimens.

Some false positives can be acquired, e.g., with the circuit digitizing and storing signals that were not associated with the physical phenomena being examined. Measurements can be averaged or selected from a series (e.g., rejecting potential false positives) to reduce the impact of false positives. Extracting the ToF from the signal can be automated, e.g., where the start of a wave is noted by monitoring for a high rate of change in the voltage produced by the coil. Performing ToF calculations on waves found in a HNSW using rate of change compared to monitoring for high amplitudes can better distinguish the HNSW from the noise and can help identify not only the start of a pulse but the end as well as other wave features. The ToF is expected to decrease at higher pressures as well as in thicker CCTs compared to their low-pressure thin counterparts. In the experiment, unusable HNSWs were removed in post-processing by using a few rejection criteria, such as the absence of a PSW (indicating an absence of interaction with the CEP), ToF lying outside selected statistical thresholds (such as 2 standard deviations). Of the 2,250 waveform samples collected, 166 acquisitions were removed.

The CCT of a cornea can affect IOP measurements, as ToF is dependent on both IOP and CCT. Thus, monitoring ToF alone may not result in a sufficiently accurate IOP prediction. HNSWs, captured from softer materials such as the cornea, produce waves with a higher variance in ToF. Due to the potential high variance in ToF there can be an overlap in ToF measurements between different CCTs. To further enhance the device's ability to detect and distinguish different corneas, each collected HNSW is represented as an image using the Short-Time Fourier Transform (STFT). The STFT is an algorithm that can be used to simultaneously present time and frequency domain information via a visual, image-like format. This image can serve as an input to a convolutional neural network (CNN) where a CCT classification task can be carried out on the transformed signals. This method of analysis can further enhance the tonometer's ability to produce HNSWs that can be representative of a CCT regardless of the IOP.

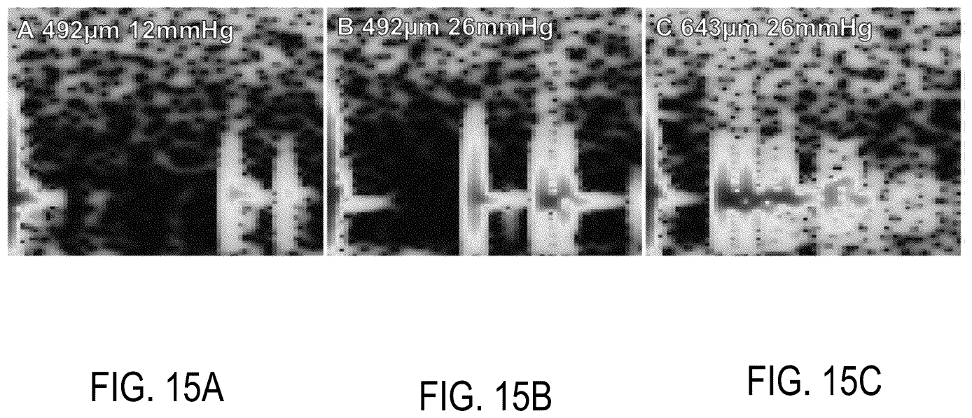

A STFT performs a Fourier Transform on a spectral window of a given signal. The spectral window is created by utilizing a function which separates the signal into corresponding time periods. The STFTs used produce a Kaiser window of length 256 and a beta value of 5. To represent the entire signal, each Kaiser window has an overlap of 220 points resulting in nearly 80 different windows. For simplification, a one-sided STFT was used as an input image of the CNN and all signals were normalized to themselves before transformation to a STFT. Both vertical and horizontal axis are removed from the image and all STFTs were cropped to include frequencies between 0-100 kHz, as that entire frequency region contains the HNSW. The image is then input into the CNN. FIGS. 15A-15B are STFTs of the same CEPs with a large difference in pressure, 12 mmHg and 26 mmHg, respectively. FIGS. 15B-15C include the STFTs from both the 492 and 643 μm CEPs but compare the cornea pressurized at 26 mmHg. Visibly, two features of the signal that stand out between images is the time separation of the first energy envelope on the far left (the ISW) to the second energy envelope (the PSW) and the power intensity associated with the PSW. As the pressure increases, the power content increases, as seen by the darker red region present in the envelope at 26 mmHg.

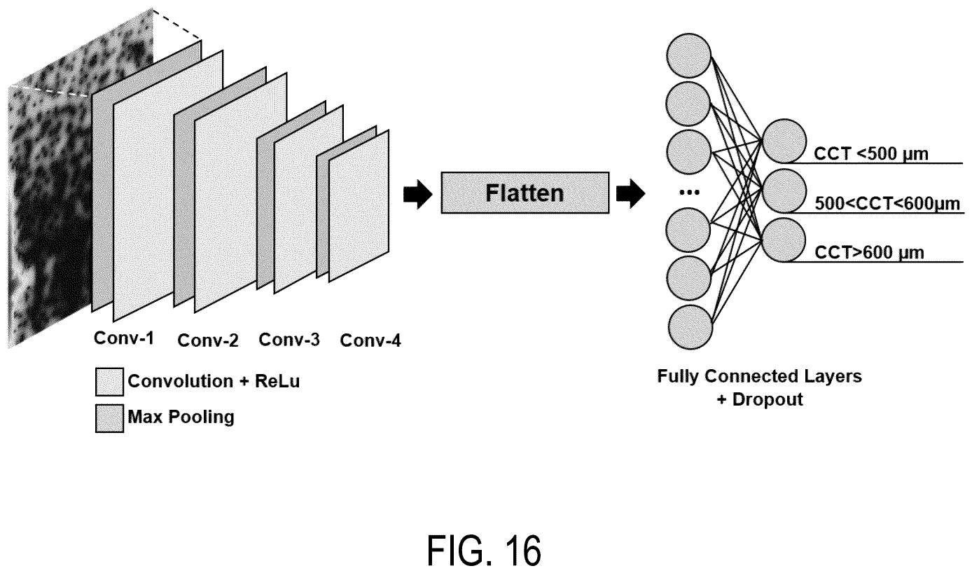

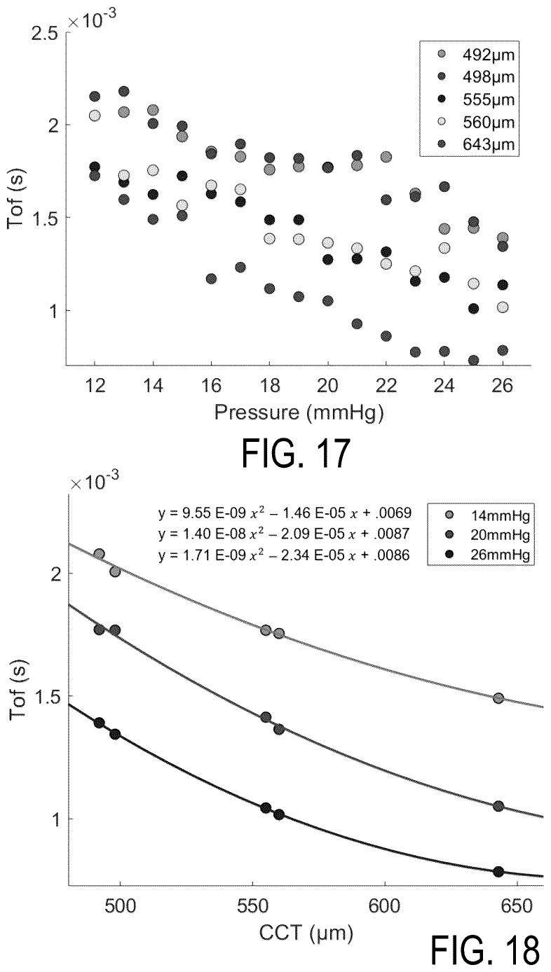

For a balanced dataset for machine learning, the five CEPs were separated into three groups: CCT<500 μm, 500 μm<CCT<600 μm, and CCT>600 μm. As there was only one CEP represented as the CCT>600 μm, data augmentation methods used in ultrasonic nondestructive evaluation were assessed. Specifically, adding noise equivalent to a SNR of 5 produced significant results when used on raw data to produce an image for classification with a CNN. Each HNSW from the 643 μm CEP underwent this noise transformation following the SNR previously used. With twice the data for CCTs>600 μm, the STFTs to be used for CCT classification were now of equal size. The train, validation, test split of each class was 70:20:10 resulting in a minimum of 819 images in each classification, 2,507 total, 500 for validation, and 253 for testing. As shown in FIG. 16, the CNN was composed of four convolution and max pooling layers using a ReLu activation function and four dense layers with dropout totaling 6.2 million trainable parameters. As discussed previously, the experimental setup and the test specimens explored the dependence of stress wave ToF on two variables: IOP and CCT. FIG. 17 is a graph showing the average ToF recorded for all five specimens with respect to pressure. For each IOP, the average of the non-rejected experimental values collected from both loading ramps was considered. As can be seen from the graph, the results are clustered in three groups based on thickness: 492 and 498 μm, 555 and 560 μm, and 643 μm. Thus, the ToF is inversely related with IOP at any given CCT. Additionally, CEPs with similar CCTs produced similar ToF trends, seen specifically with 492 and 498 μm CEPs and the 555 and 560 μm CEPs.

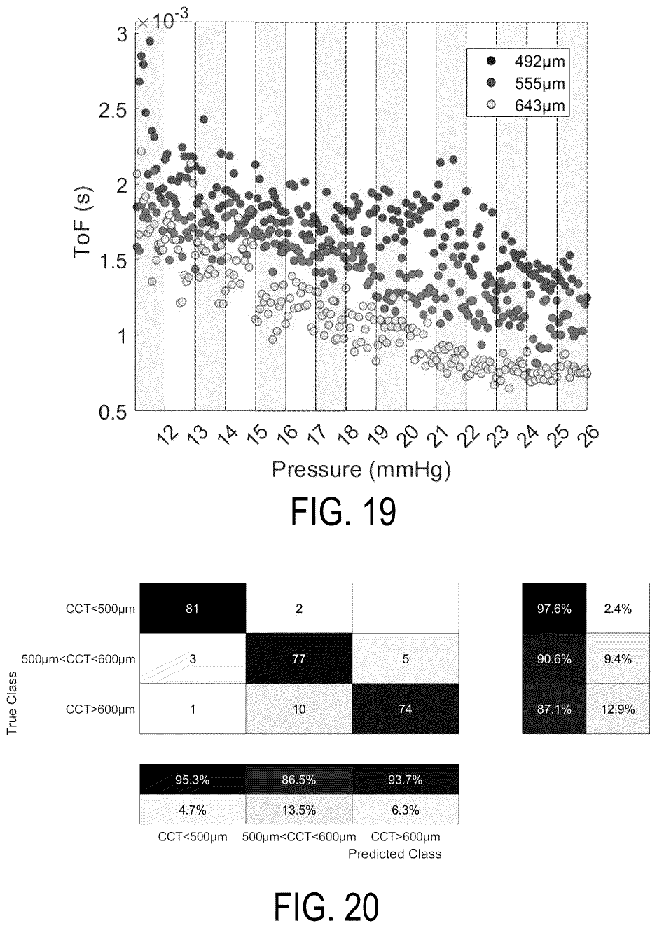

The averaged ToF calculated for each loading ramp at 14, 20, and 26 mmHg were considered and are shown in FIG. 18, which presents the corresponding ToF as a function of the CCT. A binomial interpolation was overlapped to quantify a relationship between the wave feature and the corneal thickness. Each resulting trend line had a R2 value>0.985. The variance in the ToF measurements, can be seen in FIG. 19, which shows each individual ToF measurement taken from CEPs 492 μm, 555 μm, and 643 μm. As can be seen from the figure, there is more separation of ToFs at higher pressures, but at lower pressures ToF variance is higher and overlaps with ToF measurements from other CEPs. Processing the classification task to classify the CCT of HNSW STFTs resulted in an 91.7% accuracy confirming that this device is sensitive enough to produce different HNSWs that correlate with the CCT of a CEP outside of ToF. A corresponding confusion matrix is shown in FIG. 20.

### Additional Examples

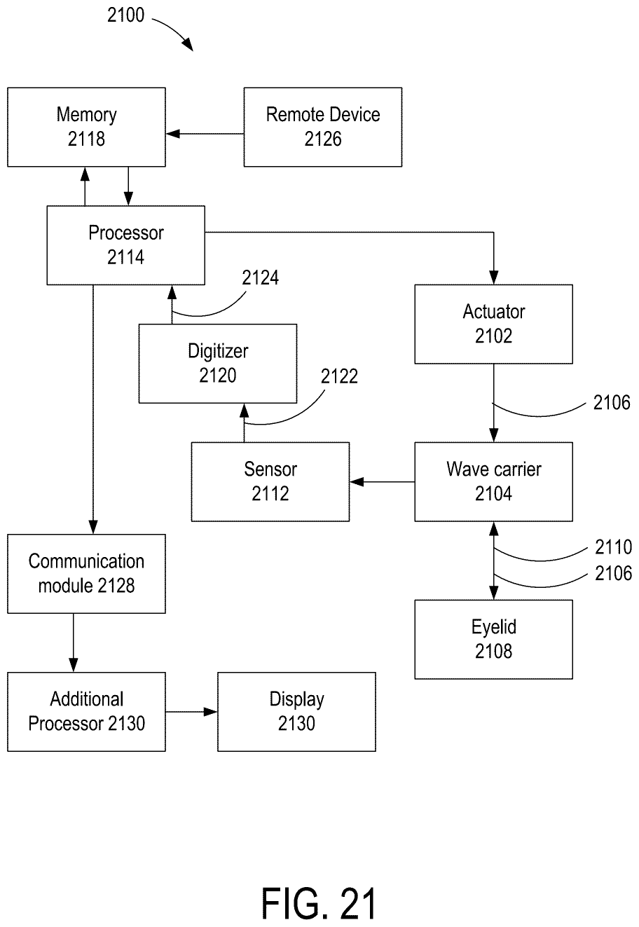

FIG. 21 shows an example system 2100 that can be used to detect intraocular pressure of an eye. The system 2100 includes an actuator 2102, such as a mechanical, electrical, or electro-mechanical actuator coupled to a wave carrier 2104, such as a chain of particles or other medium suitable to propagate a mechanical stress wave 2106, such as a solitary wave. The actuator 2102 is typically configured to strike, impact, vibrate, or otherwise induce the stress wave 2106 to propagate along the wave carrier 2104. The wave carrier 2104 is typically supported in a housing (not shown) that can support the wave carrier 2104. The housing can be used to house additional components (and related interconnections) of the system 2100 in various examples, such as the actuator 2102. The wave carrier 2104 in the housing is removably coupled to an eyelid 2108 of a patient having an IOP to be measured, such as through a membrane, arcuate or circular ridge, detent, or other support that allows transmission of the stress wave 2106 to the eyelid 2108 so that one or more reflected waves 2110 can be received by the wave carrier 2104 from the eyelid 2108. The system 2100 further includes a sensor 2112 coupled to the wave carrier 2104 and that is configured to detect characteristics of the one or more reflected waves 2110 propagating back through the wave carrier 2104 from the eyelid 2108. Various examples of the sensor 2112 can include piezoelectric sensors, magnetic coils, or any other sensor suitable for detection stress waves.

In representative examples, the system 2100 further includes a processor 1214 coupled to the actuator 2102 (or through an intermediate function generator) and configured with processor-executable instructions stored in a memory 2118 that can select and control the characteristics of the stress wave 2106 produced with the actuator 1202, such as timing, shape, amplitude, etc. Example waveforms can vary in complexity, with some examples having arbitrary shapes, others having simple on states and off states, impulses, etc. In some examples, the incident stress wave 2106 may be induced by a mechanical or electrical device that enables a mechanical impact of a striker onto the wave carrier 2104. In some examples, a digitizer 2120 is coupled to the sensor 2112 so as to receive a reflected solitary wave signal 2122 from the sensor 2112, to then produce a digitized waveform 2124 from the reflected solitary wave signal 2122 and provide the digitized waveform 2124 to the processor 2114 (or another processing unit).

In some examples, the processor 2114 is configured to determine the TOP of the eye based on the digitized waveform 2124. In some examples, estimation of IOP can be achieved by processing the digitized waveform 2124 or a transform of the waveform 2124 (such as a spectrogram) through a trained artificial neural network. In some examples, a remote device 2126 can be in communication to provide estimates for other characteristics of the eye, such as corneal thickness, eyelid thickness, etc., which can be used as additional inputs to the trained neural network to improve estimate accuracy. In further examples, a time difference between generation of the stress wave 2106 (or a suitable offset) and detection of the reflected solitary wave 2110 can be compared and a relationship between time of flight and IOP can be used to estimate the IOP.

In further examples, a communication module 2128 can receive and then transmit the digitized waveform 2124 or related detected reflected stress wave data wirelessly or through a wired communication line to an additional processor 2130 or computing unit. In some examples, the additional processor 2130 can be configured to provide additional computation or processing of the digitized waveform 2124 or related detected reflected stress wave data, such as intensive signal processing, so that the other components (such as the processor 2114) can be smaller and more streamlined (e.g., with a smaller form factor and reduced power requirements) for use in a portable stress-wave based tonometer. In further examples, the digitizer 2120 can be coupled to the processor 2114 through the communication module 2128 instead of between the processor 2114 and actuator 2102 or the processor and sensor 2112, respectively. In a particular example, the communication module 2128 communicates wirelessly to a handheld or mobile device (such as a smartphone) that includes one or more applications (“apps”) configured to provide signal processing or solitary-wave based IOP calculations and estimates. The remote device 2126 can also be coupled through the communication module 2128 or can correspond to a handheld or mobile device. In representative examples, the system includes a display 2132 that can show IOP estimates to a user of the device. As shown, the display 2132 is coupled to the additional processor 2130 but the display 2132 can also be coupled to the processor 2114, and can be situated locally, such as on the housing that houses the wave carrier 2104, or elsewhere in relation to components of the system 2100.

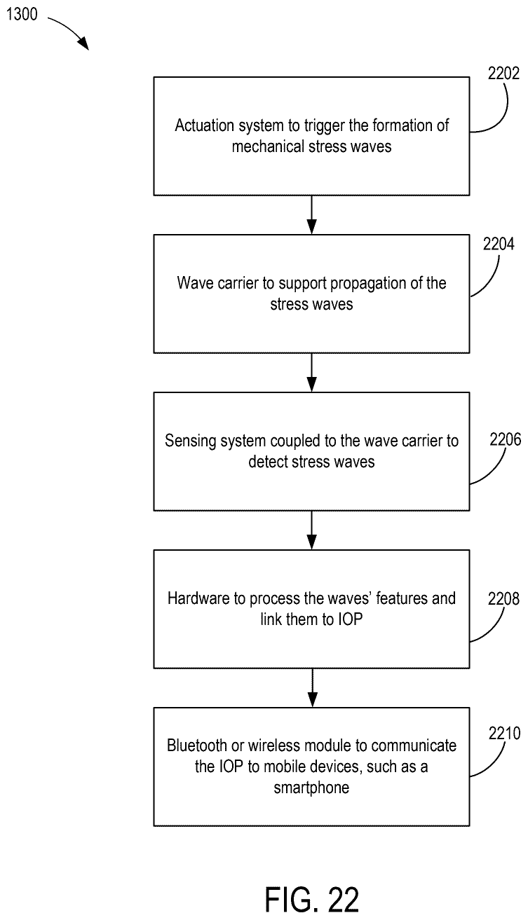

FIG. 22 shows an example tonometry system arrangement 2200 that incudes, at 2202, an actuation system configured to trigger the formation of a nonlinear solitary wave, and at 2204, a wave carrier such as a granular chain coupled to the actuation system and configured to support the propagation of stress waves such as nonlinear solitary waves. The tonometry system arrangement 2200 further includes, at 2206, a sensing system coupled to (e.g., embedded into) the wave carrier to detect the stress waves propagating through the wave carrier, including reflected stress waves, and at 2208, hardware coupled to the sensing system and configured to receive a signal associated with the detected stress wave to process the wave's features and to associate or link the features to an IOP of an eye coupled to the wave carrier. The tonometry system arrangement 2200 can also include Bluetooth or other wireless (or wired) communication modules that communicate the IOP measurement to one or more mobile devices, such as a smartphone or other smart device.

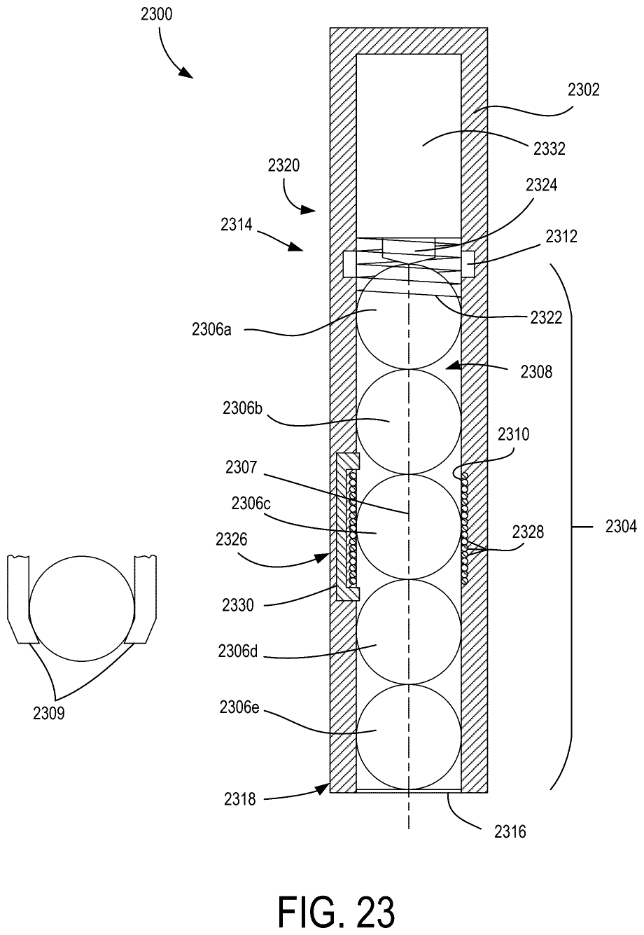

An example tonometry device 2300 is shown in FIG. 23. The tonometry device 2300 includes a housing 2302 shown in cross-section to reveal various components including internally housed components. For example, a wave carrier 2304 comprising a plurality of loosely coupled particles 2306a-2306e (or “grains”) is situated along an axis 2307 in a longitudinal interior volume 2308 defined by an interior surface 2310 of the housing 2302. In representative examples (including as shown), the particles 2306a-2306e are spherical in shape. Other shapes can be used as well provided they support the propagation of mechanical stress waves, such as nonlinear solitary waves. In selected examples, particles are cylindrical, elliptical, concave, or convex, and provide curved contact surface engagement between adjacent particles. As shown, the axis 2307 is linear, but curved, bent, forked, or other axial shapes can be provided. In various examples, the number of particles can be selected in the range of between about five and about fifty. In spherical, rectangular, and elliptical particle examples, the diameter (for spherical) or minor axis (for elliptical and rectangular) can be selected in the range of about 100 μm to 30 mm and the Young's modulus of the material forming each particle can vary from about 0.01 GPa to about 300 GPa.

The interior surface 2310 can provide a frame or support for holding the particles 2306a-2306e. The particles 2306a-2306e are loosely coupled so that the chain 2306 can partially displace along the axis 2307 after a force is received from an actuator 2312 at a first end 2314 of the wave carrier 2306. The actuator 2312 can be of any type suitable to produce a solitary stress wave along the chain 2306, such as an electromagnet, plunger, striker, etc. A flexible member 2316, such as a thin membrane, is situated at an opposite end 2318 of the wave carrier 2306 and secured to the housing 2302 (e.g., with glue) to prevent particles 2306a-2306e from exiting the interior volume 2308 or significant displacement of the chain 2306. Suitable examples of the flexible member 2316 can include aluminum or elastomer sheeting. In some examples, the particles 2306a-2306e can be retained in the interior volume 2308 with a circular or arcuate edge (or lip) 2309, e.g., as shown in the adjacent alternative version of the opposite end 2318. In representative examples, the flexible member 2316 as attached to the tonometry device 2300 can be brought into direct contact with an eyelid, or the end particle 2306e can be brought into direct contact with the eyelid, for a tonometry measurement. In some examples, a compressive member 2320 such as a spring 2322 and/or magnet 2324 can be situated at the first end 2314, the opposite end 2318, or other locations in the housing 2302 to provide a suitable compression force between the particles 2306a-2306e. Other suitable compressive members can include flexible o-rings, collars, wadding material, latches, etc.

The tonometry device 2300 can further include a stress wave detector 2326 coupled to or forming a part of at least one of the particles 2306a-2306e of the chain 2302. As shown, the stress wave detector 2326 includes a coil 2328 (shown in cross-section) encircling particle 2306c and a permanent magnet 2330 (shown in cross-section) that applies a magnetic bias across the coil 2328 in the direction of the axis 2307. In other examples, the stress wave detector 2326 can include a piezo-mechanical system. As an incident solitary wave propagates along the chain 2306 towards the opposite end 2318 and passed the stress wave detector 2326 or as a reflected solitary wave propagates along the wave carrier 2306 towards the first end 2314 and passed the stress wave detector 2326, electrical signals are produced in the coil 2328 that can be sent to additional components 2332, such as an analog-to-digital converter, waveform digitizer, and/or computing unit. The electrical signals can correspond to stress wave detection events and the signals can be converted into IOP measurement estimates. By way of example, the additional components 2332 can also include programmable measurement hardware, batteries, wireless communication modules, plugs, access ports, or other components, situated in the housing 2302. During operation the additional components 2332 can be used to produce the estimates of IOP. In selected examples, the IOP estimates can be sent, or IOP computation or other signal processing can be sent, via wireless communication (e.g., WiFi, Bluetooth, NIR, etc.) to a mobile device or other external computing device.

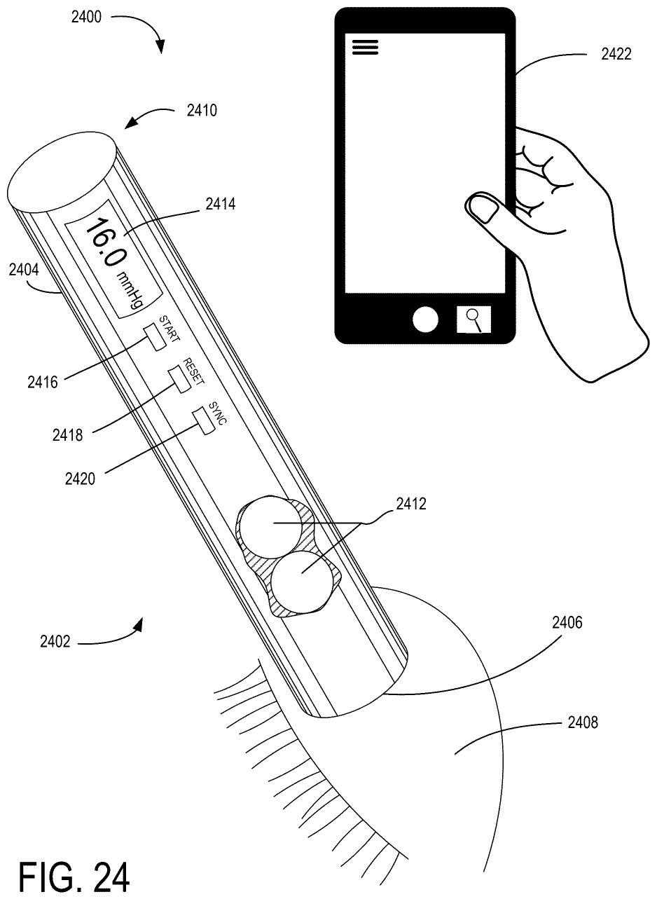

FIG. 24 shows an example tonometry system 2400 that includes a tonometer device 2402. In representative examples, the tonometer device 2402 includes a body 2404 having a cylindrical shape and a form factor similar to a pen. The body 2404 includes an application end 2406 that can be applied to an eyelid 2408 of a user and an opposite end 2410 housing various electronic circuitry. In some examples, the body 2404 has a shape that can be gripped by a user, such as at the opposite end 2410, so that the user can apply the application end 2406 to the user's eyelid to self-administer a tonometry test to produce an IOP estimate. During operation, an incident stress wave is produced within the tonometer device 2402 and directed along a wave carrier 2412 (shown in cut-away) to the eyelid 2408, and a reflected stress wave is detected with the tonometer device 2402 at a position along the wave carrier 2412. A display 2414 can situated on the body 2404 for showing the results of a tonometry test, such as by displaying an IOP estimate. One or more buttons or other interfaces, such as buttons 2416, 2418, 2420, can be situated on the body 2404 for providing various functions. For example, the button 2416 labeled “START” can be used to wake-up the tonometer device 2402 from a rest state and/or initiate a tonometry test, the button 2418 labeled “RESET” can be used to reset the tonometer device 2402 before initiation of another tonometry test, and the button 2420 labeled “SYNC” can be used to initiate communication link between the tonometer device 2402 and an external device, such as a mobile device 2422. In further examples, functionalities of different buttons can be combined or additional functions can be provided. For example, an audio or visual indication can be provided where an orientation and/or contact of device 2402 is determined to be suitable or not suitable, such as with a built-in inclinometer. In some examples, the body 2404 does not include any buttons or interfaces. In some examples, the mobile device 2422 or other external computing unit can be used to initiate and control the tonometry test.

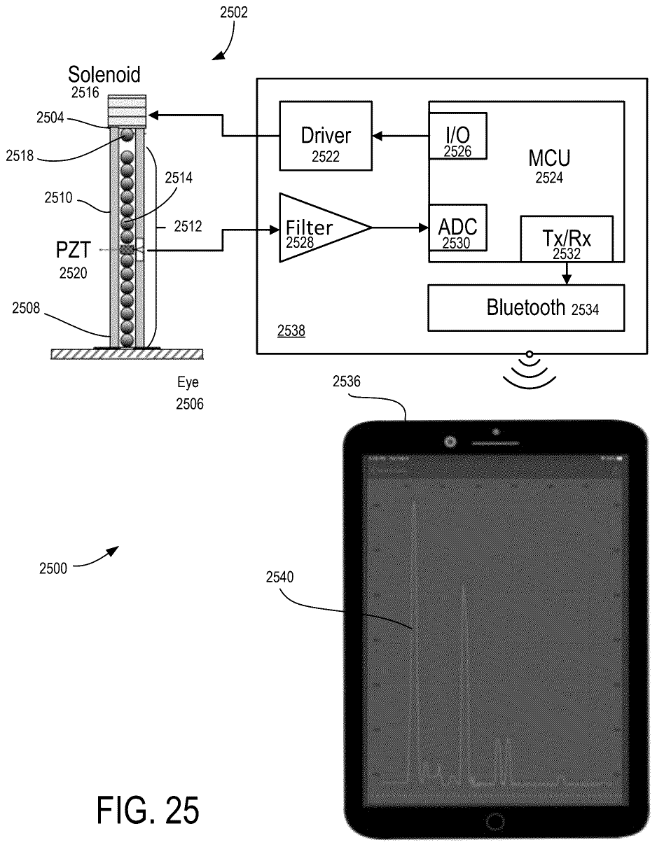

FIG. 25 shows another example of a tonometer 2500 configured to operate wirelessly in part. Some examples can include features from examples described in the article “Wireless Module for Nondestructive Testing/Structural Health Monitoring Applications Based on Solitary Waves,” by Misra, R., Jalali, H., Dickerson, S., and Rizzo, P., published May 26, 2020 in Sensors, 20, 3016. DOI: 10.3390/s20113016, which is also incorporated by reference herein. The tonometer 2500 can include a transducer 2502 configured to produce stress waves at a first end 2504 of the transducer 2502 and to direct the stress waves to an eye 2506 (or eyelid) in stress wave communication with a second end 2508 of the transducer 2502. In some examples, the transducer 2502 includes a frame 2510 supporting a stress wave carrying array 2512 of particles 2514 which can be configured in a series to transmit the solitary stress waves in forward and reverse directions along the array 2512. In particular examples, the transducer 2502 includes a solenoid 2516 at the first end 2504 configured to suspend a striker particle 2518 at a selected height and to release the striker particle 2518 to strike the array 2512 and cause a solitary stress wave to propagate along the array 2512 toward the eye 2506. It will be appreciated that other striking mechanisms may be used, including springs or other resilient members configured to release energy to the array 2512 to induce the solitary stress waves. Combinations of mechanical and electrical components may be used in some examples, such as electromagnets and springs. After reaching the eye, a return solitary stress wave is formed and propagates from the second end 2508 back along the array 2512. A sensor 2520, such as a piezoelectric transducer, is situated within the array 2512 to detect stress waves propagating passed, e.g., embedded within a particle or as a separate type of particle. In free-fall and other striker examples, the mass of the striker, such as the striker particle 2518, can be equal to the mass of the other particles 2514 of the array 2512, thereby producing a single stress wave pulse.

In a particular example, the particles 2514 of the array 2512 include a plurality of non-ferromagnetic spheres with the striker particle 2518 being ferromagnetic. The solenoid 2516 can be configured to translate the striker particle 2518 to the selected height above the array 2512 and to release the striker particle 2518 upon cessation or interruption of the current through the solenoid 2516 so that the striker particle 2518 impacts the first particle of the array 2512 to form a solitary stress wave. In the particular example, the sensor 2520 includes a lead zirconate titanate (Pb[ZrxTi1-x]O3) wafer transducer (PZT) embedded between a pair of metal disks having a diameter similar to the particles 2514. For metal disk examples, the PZT can be insulated with an insulation layer. In some examples, the combined mass of the PZT and disks can be the same as one of the particles 2514.

A driver 2522, such as a current source or other controllable driving source, is coupled to the solenoid 2516 so as to controllably provide current to the solenoid 2516 for controllable generation of solitary stress waves. The driver 2522 can be coupled to a microcontroller (MCU) 2524 through an I/O port 2526 (such as general purpose I/O (GPIO)) and the MCU 2524 can be configured with instructions to control the initiation, repetition rate, repetitions, and other characteristics of the solitary stress waves generated by driving the solenoid 2516 with the driver 2522. The solitary stress waves propagating along the array 2512 can be detected by the sensor 2520 and the sense signal produced can be directed to an analog filter 2528 and the filtered signal can be subsequently sampled by an analog to digital converter (ADC) 2530 which is typically a component part of the MCU 2524. In some wireless examples, the MCU 2524 can then send the stress wave data samples through communication port 2532 to an integrated circuit (IC) 2534 enabled for, e.g., Bluetooth Low Energy (BLE) communication using the Universal Asynchronous Receiver/Transmitter (UART) protocol. The protocol can allow for the wireless transmission of the stress wave data to another computing device 2536 capable of BLE communication, such as a handheld mobile device, laptop, tablet, etc. In some examples, the computing device 2536 is wireless coupled to transmit stress wave commands to the MCU 2524. In some examples, the computing device 2536 can be configured to display stress waves 2540 or other information, such as intraocular pressure associated with the stress wave data. In some wireless examples, the driver 2522, filter 2528, MCU 2524, and Bluetooth IC 2534 are arranged together on a printed circuit board (PCB) 2538. The PCB 2538 can be coupled to the transducer 2502 (e.g., solenoid 2516 and sensor 2520) through wired communication either through an extended wire or close together, such as within the frame 2510 of the transducer 2502. In other examples, different arrangements of wired and wireless communication can be provided, such as providing wireless communication between the driver 2522 and the MCU 2524, between the sensor 2520 and the filter 2528, and/or between the filter 2528 and the MCU 2524. In some examples, the MCU 2524 can be integrated into or form part of the computing device 2536 which can eliminate wireless communication between the MCU 2524 and the computing device 2536.

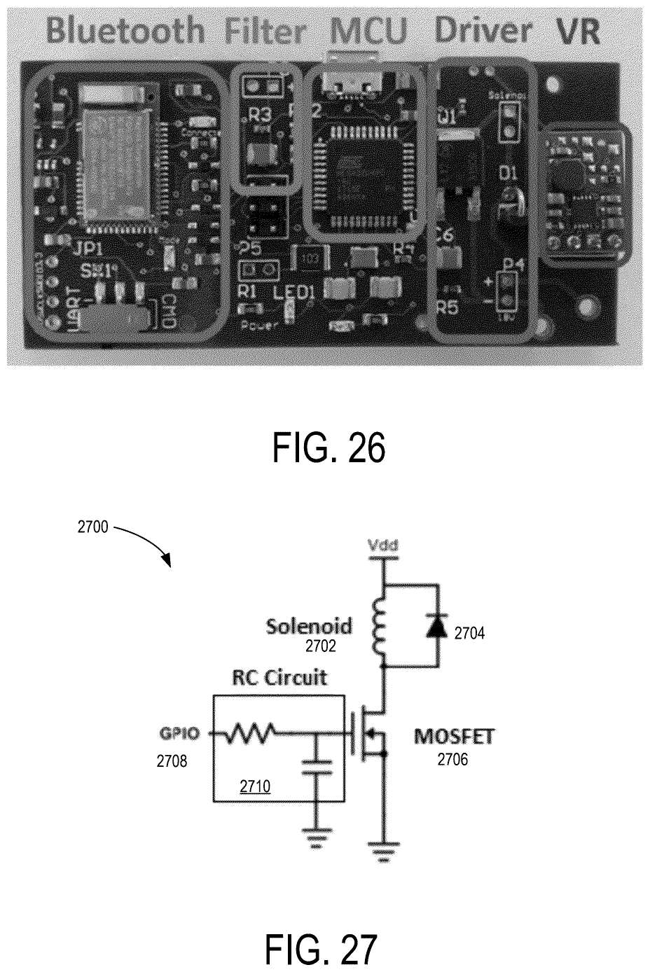

In a particular example shown in FIG. 26, the PCB 2538 had a form factor of 76.2×36.8 mm². As shown in FIG. 26, the PCB 2538 included a Bluetooth transceiver, a filter, an MCU, and a voltage regulator (VR). The MCU 2524 was an ATMega32u4 with 32 kB of flash memory for storing embedded programs, 2 kB of SRAM for storing measurement data, peripherals sufficient to induce and measure the stress wave signal, and libraries that allowed for easy communication with the Bluefruit LE module. The MCU 2524 included a universal serial bus (USB) controller, allowing for local data collection without an additional an integrated circuit to perform FTDI to UART conversion. The size of further examples can be substantially reduced further such that the PCB 2538 or related driving and sensing components can be packaged with the transducer 2502 to form a singular handheld device with various capabilities. For example, some examples can control and store measurement data, with some examples allowing accessibility and/or display of the measurement data by a separate computing device, such as a mobile device, laptop, tablet, etc. Some examples can control, store, and display measurement data, with or without accessibility by a separate computing device.

In some examples, actuation can be effected with power supplied by batteries rather than through a bulky external power supply. In some examples, DC current used to drive the electromagnet of the solenoid 2516 can be supplied through the PCB 2538. Similar to some wired examples, in a wireless example the solenoid 2516 is energized for 250 ms, which corresponds to an interval of sufficient duration to lift the striker particle 2518 until it touches the electromagnet before falling freely onto the array 2512. The energy necessary to deliver the current necessary to operate the electromagnet is significant with respect to the other electronic components of the tonometer 2510 and is directly proportional to the weight and the falling height of the striker particle 2518. To supply the necessary energy, an example power source for the solenoid and driver circuit allows the control of the striker while maintaining portability. For example, LiPo, Li-Ion, or other suitable energy dense batteries may be used to provide a sufficient discharge rate and storage capacity for solitary stress wave IOP measurements. Shorter duration and/or smaller energy consumptions can be obtained by decreasing the falling height of the striker, by making the striker lighter (in order to be able to use smaller solenoids), or by minimizing the friction between the striker and the inner wall of the guide, by way of example.

FIG. 27 shows an example control circuit 2700 for providing actuation of a solenoid 2702, which can be used with various examples herein including the driver 2522 of tonometer 2500. A 1N4003 diode 2704 is situated in parallel with the solenoid 2702, which operates as a flyback diode that prevents a voltage spike resulting from turning off the solenoid 2702, from damaging a metal-oxide semiconductor field-effect transistor (MOSFET) 2706, which might otherwise reduce product lifespan and reliability. The MOSFET 2706 operates as an open circuit with a GPIO pin 2708 (or other control circuit input) in an off state, and operates as a closed circuit with the GPIO pin 2708 in an on state, allowing for the control of the current through the solenoid 2702 via, e.g., software. In an example, the MOSFET 2706 was an NTD3055-150 from ON Semiconductor, which is configured to operate in low voltage, high-speed switching applications in power supplies, converters and power motor controls and bridge circuits. An RC circuit 2710 at the gate of the transistor 2706 provides a slight delay between turning the GPIO pin 2708 off in software and the moment at which a magnetic striker particle on top of a tonometer chain drops. Various resistor and capacitor values may be used to adjust the time constant of the RC circuit 2710. In representative examples, the time constant is selected to be at least three times larger than the minimum delay that an MCU and/or related electronics can produce. This prevents an undesirable scenario where the MCU samples an ADC after an incident solitary stress wave passes the sensor configured to detect the wave. The delay introduced by the RC circuit 2710 also safeguards against similar detection failures where mechanical adjustments to the transducer are made that can reduce the amount of time it takes for the striker to fall. In one example, the RC circuit 2710 consisted of a 10 kΩ resistor and a 33 nF capacitor, resulting in a time constant of 333 μs.

In representative examples, the filter 2528 can be selected as a passive low-pass filter that can be used to remove white noise and provide anti-aliasing. The cutoff frequency can be determined by examining the frequency spectrum of solitary stress waves recorded at a selected sampling rate (such as 2 MHz) by placing a transducer above various surfaces. In one example, a 12.7 mm thick steel plate was used. Example filters can provide a cutoff frequency at a frequency selected to provide noise rejection as well as to retain significant solitary stress wave information. Such a cutoff frequency position can also serve to provide antialiasing. Example cutoff frequencies can include 10 kHz, 50 kHz, 100 kHz, 500 kHz, 1 MHz, 2 MHz, 10 MHz, 100 MHz, etc. In an example, the components of the 2528 filter have values equal to 2Ω and 33 nF, resulting in a cutoff frequency of 2.411 MHz. However, it will be appreciated that the filter and related characteristics can be modified based upon further refinements of the application of the solitary stress waves to tonometry, including variations in the characteristics of transducers, electronic componentry, the particles in the array, the properties of the eye (including intervening elements such as an eyelid) to be monitored, and the duration of the incident and reflected waves. In representative examples, the circuit coupling between the sensor 2520 and associated wiring on one side and the MCU 2524 and the signal sensing components, including the filter 2528 and ADC 2530, on the other side, can be impedance matched to reduce electrical reflections and thereby maximize signal quality.

While the PCB 2538 discussed above uses a Bluetooth module and associated communication protocol for communication between the tonometer MCU 2524 and the external mobile device 2536, it will be appreciated that other wireless protocols may be used. For short-distance communication, Bluetooth protocol is beneficial in view of its compatibility with a substantial variety of electronic devices, including consumer devices such as smartphones, tablets, and laptops. Additionally, Bluetooth communication does not rely on any external network. In typical examples, the Bluetooth LE UART module relies on the general-purpose, ultra-low power System-on-Chip nrF51822 to provide wireless communication with any BLE-compatible device. The term “System-on-Chip” means that the nrF51822 is a complete computer system within a single chip that can act independently from the MCU. This capability can allow for improvements to future iterations of the PCB 2538. The nrF51822 has the ability to choose between UART and SPI communication with external devices, and sleep modes for power preservation.

Software applications can be configured so that the mobile device can communicate with the transducer 2502 of the tonometer 2500 via the PCB 2538. In a selected example, a software application was adapted from a general application framework and customized by added a data streaming mode capable of compartmenting the data it received from different solitary stress wave runs into separate graphs. These plots can also be exported as data files for further processing. The data streaming was designed to work with the messaging protocol programmed into the MCU 2524. In representative examples, the software provides a list of Bluetooth devices within the vicinity. After the user selects the appropriate device, the “Data Stream” menu option allows the user to remotely drive the striker of the tonometer 2510 and to collect data from the embedded sensor disk. Selecting a “Data Stream” option prompts the user to select the number of strikes and the length (data points) of the signal. After the PCB 2538 receives the command, it actuates the transducer 2502, collects samples of the time waveform from the ADC 2530, sends the data to the mobile device 2536, and iterates the process as many times as the number of strikes chosen by the user. During the process, the waveforms can be displayed in real-time on the smart device. The software application and the 2538 PCB together can define a self-contained tonometry system that only requires a basic knowledge of smart mobile devices to operate.

In a particular implementation of the tonometer 2500, the ADC 2530 within the AtMega32u4 was used to digitize the signals detected by the embedded sensor disk 2520. The clock of the ADC 2530 was set equal to 1 MHz, as setting the clock to a higher frequency would reduce the resolution for this particular device. A single conversion takes 13 clock cycles, and the clock frequency was set to 16 MHz, so the highest theoretically achievable sampling frequency was 1 (MHz)/13=77 kHz. The ADC 2530 uses a sample-hold capacitor, which is first charged by the signal and then closed-off from the input signal so that the voltage of the signal at that time can be indirectly read through the voltage on the capacitor at that moment. A 5 V power supply for the ATMega32u4 and the Bluetooth module was generated with a 3.7 V single-cell LiPo and a Pololu 5 V Step-Up Voltage Regulator U1V11F5. The U1V11F5 can handle input voltages in a range of 1 to 5.5 V, so it is robust to small voltage drops caused by the discharging of the single-cell LiPo. The PCB 2538 follows a protocol for collecting data and sending the data wirelessly to the computing device 2536. After the first time the PCB 2538 is turned on, it waits for a mobile device to connect to it. After a device has connected, the PCB 2538 turns the solenoid 2516 on and off again, starts a timer, and then collects samples from the ADC 2530 until the ADC 2530 reading passes a certain threshold. This allows the PCB 2538 to learn the timing between the dropping of the striker particle 2504 and observing a HNSW. It then allows the wirelessly coupled computing device app to send to the PCB 2538 the desired number of samples and runs after which it executes the appropriate number of runs while recording the desired number of samples in time for each run. In some examples, IOP measurements and related data can be computed and displayed on the computing device 2536 after completion of the test.

Further examples are described in U.S. Pat. No. 11,957,413 (incorporated by reference herein) which can be configured to implement any of the techniques described herein or include any of the features described herein.

### General Considerations

As used in this application and in the claims, the singular forms “a,” “an,” and “the” include the plural forms unless the context clearly dictates otherwise. Additionally, the term “includes” means “comprises.” Further, the term “coupled” does not exclude the presence of intermediate elements between the coupled items.

The systems, apparatus, and methods described herein should not be construed as limiting in any way. Instead, the present disclosure is directed toward all novel and non-obvious features and aspects of the various disclosed embodiments, alone and in various combinations and sub-combinations with one another. The disclosed systems, methods, and apparatus are not limited to any specific aspect or feature or combinations thereof, nor do the disclosed systems, methods, and apparatus require that any one or more specific advantages be present or problems be solved. Any theories of operation are to facilitate explanation, but the disclosed systems, methods, and apparatus are not limited to such theories of operation.

Although the operations of some of the disclosed methods are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed systems, methods, and apparatus can be used in conjunction with other systems, methods, and apparatus. Additionally, the description sometimes uses terms like “produce” and “provide” to describe the disclosed methods. These terms are high-level abstractions of the actual operations that are performed. The actual operations that correspond to these terms will vary depending on the particular implementation and are readily discernible by one of ordinary skill in the art.

In some examples, values, procedures, or apparatus' are referred to as “lowest,” “best,” “minimum,” or the like. It will be appreciated that such descriptions are intended to indicate that a selection among many used functional alternatives can be made, and such selections need not be better, smaller, or otherwise preferable to other selections.

Algorithms may be, for example, embodied as software or firmware instructions carried out by one or more digital computers. For instance, any of the disclosed stress wave tonometry techniques can be performed by a computer or other computing hardware (e.g., an ASIC or FPGA) that is part of a tonometry system. The tonometry system can be connected to or otherwise in communication with the stress wave detector and be programmed or configured to receive detected stress wave characteristics and perform intraocular pressure measurement and estimate computations (e.g., any of the tonometry techniques disclosed herein). The computer can be a computer system comprising one or more processors (processing devices) and tangible, non-transitory computer-readable media (e.g., one or more optical media discs, volatile memory devices (such as DRAM or SRAM), or nonvolatile memory or storage devices (such as hard drives, NVRAM, and solid state drives (e.g., Flash drives)). The one or more processors can execute computer-executable instructions stored on one or more of the tangible, non-transitory computer-readable media, and thereby perform any of the disclosed techniques. For instance, software for performing any of the disclosed embodiments can be stored on the one or more volatile, non-transitory computer-readable media as computer-executable instructions, which when executed by the one or more processors, cause the one or more processors to perform any of the disclosed tonometry techniques. The results of the computations can be stored (e.g., in a suitable data structure or lookup table) in the one or more tangible, non-transitory computer-readable storage media and/or can also be output to the user, for example, by displaying, on a display device (such as a display on the housing of a device directing the stress wave to the eye or remotely on a mobile device or other display), detected wave characteristics or intraocular pressures with a graphical user interface.

Having described and illustrated the principles of the disclosed technology with reference to the illustrated embodiments, it will be recognized that the illustrated embodiments can be modified in arrangement and detail without departing from such principles. For instance, elements of the illustrated embodiments shown in software may be implemented in hardware and vice-versa. Also, the technologies from any example can be combined with the technologies described in any one or more of the other examples. It will be appreciated that procedures and functions such as those described with reference to the illustrated examples can be implemented in a single hardware or software module, or separate modules can be provided. The particular arrangements above are provided for convenient illustration, and other arrangements can be used.

In view of the many possible embodiments to which the principles of the disclosed technology may be applied, it should be recognized that the illustrated embodiments are only representative examples and should not be taken as limiting the scope of the disclosure. Alternatives specifically addressed in these sections are merely exemplary and do not constitute all possible alternatives to the embodiments described herein. For instance, various components of systems described herein may be combined in function and use. We therefore claim all that comes within the scope of the appended claims.

We claim:

1. A method, comprising:
receiving tonometry data from a tonometer device, the data including time domain and frequency domain information; and
estimating an intraocular pressure of an eye by processing the tonometry data through a machine learning model trained on pre-existing tonometry data and intraocular pressure data associated with the pre-existing tonometry data.

2. The method of claim 1, wherein the tonometry data comprises stress wave data and the pre-existing tonometry data comprises pre-existing stress wave data.

3. The method of claim 1, wherein the pre-existing tonometry data includes pre-existing corneal thickness data, wherein tonometry data includes corneal thickness measurement data associated with the eye, wherein the estimating includes processing the tonometry data including the corneal thickness measurement data associated with the eye through the trained machine learning model.

4. The method of claim 1, wherein the pre-existing tonometry data includes pre-existing eyelid thickness data, wherein tonometry data includes eyelid thickness measurement data associated with the eye, wherein the estimating includes processing the tonometry data including the eyelid thickness measurement data associated with the eye through the trained machine learning model.

5. The method of claim 1, wherein the trained machine learning model comprises a convolutional neural network.

6. The method of claim 1, wherein the time and frequency domain information comprise one or more spectrograms.

7. The method of claim 1, further comprising:
positioning an end of a tonometer device proximate the eye;
directing one or more incident tonometer waves to the eye along a wave carrier and receiving one or more return tonometer waves from the eye; and
detecting at least the one or more return tonometer waves with a tonometer sensor to produce a tonometer signal associated with the tonometry data.

8. The method of claim 7, wherein the directing comprises directing one or more incident solitary stress waves to the eye, and the detecting comprises detecting at least a primary reflected solitary stress wave and a secondary solitary stress wave for each incident solitary stress wave directed to the eye.

9. The method of claim 7, wherein the directing comprises producing the one or more incident solitary waves in the wave carrier with an actuator.

10. The method of claim 9, wherein the actuator comprises a striker.

11. The method of claim 7, wherein the positioning comprises contacting the end of the tonometer device to an eyelid of the eye.

12. The method of claim 7, wherein the end comprises a flexible membrane configured to directly contact the eye or eyelid of the eye.

13. The method of claim 7, wherein the end comprises a retained end particle of a chain of particles comprising the wave carrier, wherein the end particle is configured to directly contact the eye or eyelid of the eye.

14. The method of claim 7, wherein the directing comprises directing the one or more incident tonometer waves to the eye through the eyelid.

15. The method of claim 1, further comprising:
reducing electrical reflections that deteriorate the tonometer signal received by a microcontroller of the tonometer device by providing an impedance matching between the microcontroller and an electrical circuit coupling the tonometer sensor to the microcontroller.

16. The method of claim 1, further comprising:
determining a suitability of an alignment of the tonometer device in relation to the eye before performing a tonometer measurement, by detecting an orientation of the tonometer device in relation to Earth's gravitational field using an inclinometer of the tonometer device.

17. The method of claim 16, wherein the determining the suitability of the alignment comprises determining whether wave carrying and striking components of the tonometer device are aligned within a range parallel to Earth's gravitational field.

18. The method of claim 16, further comprising preventing the performing of a measurement where the alignment is determined to be not suitable.

19. The method of claim 16, further comprising providing an audio and/or visual indication before and/or after the alignment is determined to be suitable.

20. The method of claim 1, further comprising training the machine learning model on the pre-existing tonometry data and the intraocular pressure data associated with the pre-existing tonometry data.

21. An apparatus, comprising a tonometer configured to perform the method of claim 1.

22. An apparatus, comprising:
a wave carrier configured to propagate one or more incident stress waves to an eye;
a housing configured to support the wave carrier;
a sensor coupled to the wave carrier and configured to detect one or more return stress waves propagating along the wave carrier from the eye; and
a processor configured to receive stress wave tonometry data from the sensor, the data including time domain and frequency domain information, wherein the processor is configured estimate an intraocular pressure of the eye based on both the time domain and frequency domain information.

23. The apparatus of claim 22, wherein the processor is configured to estimate the intraocular pressure by processing the stress wave tonometry data through a machine learning model trained on pre-existing tonometry data and intraocular pressure data associated with the pre-existing tonometry data.

24. The apparatus of claim 22, wherein the wave carrier comprises a particle array, wherein the particle array comprises a plurality of adjacently arranged loosely coupled particles that propagate the incident and return stress waves from one particle to the next.

25. The apparatus of claim 24, further comprising a particle array compressive member coupled to at least one of the particles to provide a compression for the particle array that contact among the particles.

26. The apparatus of claim 24, wherein the particles have spherical, cylindrical, or elliptical shape, or a mix of shapes, and are made of PTFE, steel, or another material having an elastic modulus between 0.01 and 200 GPa.

27. The apparatus of claim 24, wherein the sensor comprises a magnetic coil encircling at least a portion of at least one of the particles or a piezoelectric transducer embedded in at least one of the particles.

28. The apparatus of claim 22, wherein the sensor comprises a stress wave sensor.

29. The apparatus of claim 22, further comprising a retaining support configured to retain the wave carrier in the housing and to allow an end of the wave carrier to become removably coupled to the eyelid of the eye.

30. The apparatus of claim 29, wherein the retaining support comprises a membrane attached to the housing.

31. The apparatus of claim 29, wherein the retaining support comprises an arcuate or circular ridge.

32. The apparatus of claim 29, wherein the retaining support is configured to allow an end of the wave carrier to directly contact the eyelid.

33. The apparatus of claim 22, further comprising an actuator coupled to the particle array and configured to produce the incident stress wave in the wave carrier.

34. The apparatus of claim 33, further comprising:
driving circuitry configured to drive the actuator wherein the driving circuitry includes delay circuitry configured to reduce a sampling error; and
filter circuitry configured to filter stress wave data detected by the sensor.

35. The apparatus of claim 32, further comprising circuitry configured to wirelessly transmit the filtered stress wave data to a separate computing device.

36. The apparatus of claim 33, wherein the actuator comprises a solenoid configured to raise a striker particle and to drop the striker particle from a height.

37. The apparatus of claim 33, further comprising:
a digitizer coupled to the sensor and configured to digitize the detected return stress wave to form a digitized return solitary wave signal;
a processor coupled to the digitizer and function generator; and
a memory coupled to the processor and configured with instructions executable by the processor for controlling the generation of the incident stress wave in the wave carrier.

38. The apparatus of claim 37, wherein the memory is further configured with instructions for determining an intraocular pressure of an eye based on one or more characteristics of the digitized return stress wave signal.

39. The apparatus of claim 37, further comprising a wireless communication node coupled to the processor and configured to communicate data describing the digitized return stress wave signal to an external signal processing device.

40. A method, comprising:
directing an incident stress wave along a wave carrier coupled to an eye;
detecting at least one return stress wave propagating along the wave carrier from the eye; and
producing a detected stress wave signal including time domain and frequency domain information.

41. The method of claim 40, further comprising estimating an intraocular pressure of the eye by processing the stress wave tonometry data of the stress wave signal through a machine learning model trained on pre-existing tonometry data.

42. The method of claim 39, further comprising estimating an intraocular pressure by comparing characteristics of the stress wave signal to a relationship between a time of return stress wave time of flight and/or a ratio of incident and detected wave amplitudes and a correlated intraocular pressure.

43. A computer-readable medium including stored instructions which, when executed by one or more computing devices, cause the computing devices to estimate intraocular pressure according to claim 1.

44. The computer readable medium of claim 43, further comprising stored instructions causing the computing devices:
to direct an actuator to produce an incident stress wave along a wave carrier coupled to the eye, and
to store the stress wave data including data from a detection signal received in response to the actuating.

45. A tonometer, comprising:
a tonometer wave carrier arranged to propagate one or more waves to an eye;
a tonometer sensor coupled to the wave carrier to detect characteristics of one or more return waves received in response to the one or more waves that propagate to the eye, and a microcontroller circuit electrically coupled to the sensor to receive an electrical signal from the sensor, wherein the electrical signal has characteristics based on the detected one or more return waves, wherein the electrical coupling between the microcontroller circuit and the sensor is impedance matched to reduce electrical reflections that reduce a quality of the electrical signal received by the microcontroller.

46. The tonometer of claim 45, wherein the microcontroller circuit includes a low pass filter and an analog to digital converter and the sensor includes a sensing element and wiring coupling the sensor to the microcontroller circuit.

47. An apparatus, comprising:
a wave carrier configured to propagate one or more incident waves to an eye;
a housing configured to support the wave carrier and be held by a user to measure an intraocular pressure of the eye;
a sensor coupled to the wave carrier and configured to detect one or more return waves propagating along the wave carrier from the eye; and
a sensor coupled to the wave carrier and housing and configured to detect an orientation of the wave carrier in relation to Earth's gravitational field, wherein the detected orientation is used to provide the user with an indication of suitability and/or non-suitability of the orientation for intraocular pressure measurement.
